(19) United States
(12) Patent Application Publication
(10) Pub. No.: US 2008/0255066 A1
(43) Pub. Date: Oct. 16, 2008

(54) ANTISENSE OLIGONUCLEOTIDE STRATEGIES FOR THE ENHANCEMENT OF CANCER THERAPIES

(75) Inventors: D. JAMES KOROPATNICK, London (CA); Mark D. Vincent, London (CA)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Sarissa Inc., London (CA)

(21) Appl. No.: 12/029,297

(22) Filed: Feb. 11, 2008

Related U.S. Application Data
(63) Continuation-in-part of application No. 10/510,899, filed on Jun. 22, 2005, now abandoned.
Application No. 10/510,899 is a continuation-in-part of application No. 10/597,409.
Application No. 10/597,409 is a continuation-in-part of application No. 11/908,389.
Application No. 11/908,389 is a continuation-in-part of application No. 11/987,568, filed on Nov. 30, 2007.
Application No. 11/987,568 is a continuation-in-part of application No. 10/597,409.
Application No. 10/597,409 is a continuation-in-part of application No. 11/171,435, filed on Jul. 1, 2005, now abandoned.
Application No. 11/171,435 is a continuation of application No. 09/509,418, filed on Jul. 11, 2000, now abandoned.
(60) Provisional application No. 60/538,886, filed on Jan. 23, 2004.
(60) Provisional application No. 60/556,368, filed on Mar. 26, 2004.
(60) Provisional application No. 60/661,993, filed on Mar. 11, 2005.
(60) Provisional application No. 60/911,759, filed on Apr. 13, 2007.

(30) Foreign Application Priority Data
Sep. 23, 1997 (GB) 9720107.3
Oct. 17, 1997 (GB) 9722012.3
Jun. 6, 1998 (GB) 9812140.3
Apr. 8, 2002 (CA) 2,380,970

(51) Int. Cl. A61K 31/7105; A61P 35/00

(52) U.S. Cl. 514/44

(57) ABSTRACT

Effective combinations of antisense agents directed against thymidylate synthase mRNA are provided for use in cancer therapies. Combinations of antisense agents have enhanced activity compared to the activity of the individual antisense agents when used alone. The combinations may be used in conjunction with one or more chemotherapeutic agents to enhance the effects of the chemotherapeutic(s). Such antisense agent combinations constitute improved antisense therapies with application to a variety of cancers or proliferative disorders, including drug resistant cancers.

### CROSS REFERENCE TO RELATED APPLICATIONS

This application is a continuation-in-part application of U.S. patent application Ser. No. 10/510,899, filed Jun. 22, 2005, which is a U.S. national phase application of International Patent Application No. PCT/CA03/00480, filed Apr. 8, 2003, which claims priority to Canadian Patent Application No. 2,380,970, filed Apr. 8, 2002. This application is also a continuation-in-part application of U.S. patent application Ser. No. 10/597,409, filed Jul. 24, 2006, which is a U.S. national phase application of International Patent Application No. PCT/CA05/00069, filed Jan. 24, 2005, which claims priority to U.S. Provisional Application No. 60/538,886, filed Jan. 23, 2004 and U.S. Provisional Application No. 60/556,368, filed Mar. 26, 2004. This application is also a continuation-in-part application of U.S. patent application Ser. No. 11/908,389, filed Mar. 13, 2006, which is a U.S. national phase application of International Patent Application No. PCT/CA06/00350, filed Mar. 13, 2006, which claims priority to U.S. Provisional Application No. 60/661,993, filed Mar. 11, 2005. This application is also a continuation-in-part application of U.S. Patent Application No. not yet available (an application having Attorney Docket No. 753-109CIP; Inventors: KOROPATNICK, Donald James and VINCENT, Mark David; and entitled “Antisense Oligonucleotides Against Thymidylate Synthase”), filed Nov. 30, 2007, which is a continuation in part of U.S. patent application Ser. No. 10/597,409, filed Jan. 24, 2005 and U.S. patent application Ser. No. 11/171,435, filed Jul. 1, 2005, said Ser. No. 11/171,435 is a continuation of U.S. patent application Ser. No. 09/509,418, filed Sep. 17, 1998, which is a U.S. national phase application of International Patent Application No. PCT/GB98/02820, filed Sep. 17, 1998, which claims priority to British Patent Application No. 9722012.3 filed Oct. 17, 1997, British Patent Application No. 9720107.3 filed Sep. 23, 1997 and British Patent Application No. 9812140.3 filed Jun. 6, 1998 and U.S. Provisional Application No. 09/509,418 filed Sep. 17, 1998. This application also claims the benefit of U.S. Provisional Application No. 60/911,759 filed Apr. 13, 2007. The disclosures of each of the above-identified patent applications is hereby incorporated by reference in their entireties, including all sequences, tables and figures, for any and all purposes.

### FIELD OF THE INVENTION

The present invention pertains to the field of antisense oligonucleotides, in particular to combinations of antisense oligonucleotides for use in cancer therapies.

### BACKGROUND

The use of antisense oligodeoxynucleotides (ODNs) as therapeutic molecules is known. Several antisense ODNs targeting a variety of molecules have antiproliferative effects against neoplastic cells in vitro and in vivo (Gewirtz, 2000, J. Clin. Oncol. 18:1809-1811), and several have demonstrated anti-tuniour activity and limited toxicity in Phase I clinical trials (Smith and Wickstrom, 2000, Methods Enzymol. 314:537-580). Typically an antisense ODN therapeutic comprises a single ODN targeting a specific mRNA, however a handful of studies have examined the use of multiple antisense ODNs, each ODN designed to target a distinct mRNA. Such multiple antisense ODNs targeting separate mRNAs have been reported to exhibit cooperative, or more than additive, effects (Normanno et al., 1996, Clin. Cancer Res. 2:601-609; Skorski et al., 1995, J. Exp. Med. 182:1645-1653; Skorski et al., 1996, Blood 88:1005-1012). For example, antisense ODNs targeting three members of the epidermal growth factor family (cripto, amphiregulin, and transforming growth factor α) were used in various combinations against human colon carcinoma cells in vitro. The combination of all three ODNs was shown to be particularly effective in the inhibition of anchorage-independent growth of the tumour cells (Normanno et al., 1996, Clin. Cancer Res. 2:601-609).

A combination therapy using antisense ODNs targeting BCR-ABL and c-myc has been reported that was more effective than treatment with one ODN alone in delaying tumour growth and prolonging survival in a chronic myelogenous leukemia model in vitro and in vivo (Skorski et al., 1995, J. Exp. Med. 182:1645-1653; Skorski et al., 1996, Blood 88:1005-1012). When the antisense ODNs against these two targets were combined, specific decreases in both target mRNAs correlated with decreased hematopoeitic colony-forming ability in vitro and substantially prolonged the survival of tumour-bearing mice compared with either antisense ODN alone (Skorski et al., 1996, Blood 88:1005-1012).

In other studies, however, the co-operative effects of antisense ODNs were not clearly demonstrated (Sato et al., 2000, Anticancer Res. 20:837-842; Traidej et al., 2000, Antisense Nucleic Acid Drug Dev. 10:17-27).

In the case of oligodeoxyribonucleotides, tandem targeting of three 14-mers to 10 contiguous regions of dihydrofolate reductase (DHFR) mRNA was shown to synergistically and selectively inhibit DHFR synthesis (Maher III, L, J. and Dolnick, B. J., 1988, Nucleic Acids Research. 16(8):3341-3358). The use of tandem shorter anti-sense molecules which are complementary to contiguous, non-overlapping sequences of target nucleic acids is also described in PCT Publication No. WO 98/37240.

A number of genes that express proteins implicated in cancers have been targeted by cancer therapies using chemotherapeutics. An example is the gene encoding thymidylate synthase (TS). TS is an essential enzyme in de novo production of thymidylate (Carreras and Santi, 1995, Annu. Rev. Biochem. 64:721-762) and, due to its crucial role in DNA synthesis and cell proliferation, has been an important target for cancer chemotherapy for many years (Danenberg, 1977, Biochim. Biophys. Acta 473:73-92; Danenberg et al., 1999, Semin. Oncol. 26:621-631).

Drugs that inhibit TS, such as 5-fluorouracil (5-FU) and its variants, and raltitrexed (Tomudex®), have become integral drugs in standard treatments for colorectal cancer (Papamichael, 1999, Oncologist. 4:478-487). Although reasonably successful in clinical use, both of these drugs suffer from problems of dose-limiting toxicity and outgrowth of resistant cells, motivating the continued search for alternative treatments, such as antisense ODNs that target and impact upon the expression of TS mRNA (U.S. Pat. No. 6,087,489, WO 99/15648 and WO 98/49287). A specific antisense oligonucleotide targeting the 3′ untranslated region of TS mRNA, has been shown to down-regulate the expression of TS, inhibit neoplastic cell proliferation (Berg et al., 2001, J. Pharmacol. Exp. Ther. 298:477-484, herein incorporated by reference) and sensitise HeLa cells to raltitrexed, 5-FU and 5-fluorodeoxyuridine (5-FUdR) (Ferguson, 1999, Br. J. Pharmacol. 127:1777-1786, herein incorporated by reference).

More recently, a preliminary observation that a combination of antisense ODNs complementary to TS appeared to elicit a more than additive antiproliferative effect compared to the individual antisense ODNs alone. In addition, the combination of ODNs appeared to augment the cytotoxicit of a combination treatment of raltitrexed and 5-FUdR on HeLa cells compared to the effect exerted by one of the antisense oligonucleotides alone, i.e. the specific antisense oligonucleotide discussed above (Berg et al., AACR (2001), Abstract #3903, herein incorporated by reference).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide antisense oligonucleotide strategies for the enhancement of cancer therapies. In accordance with one aspect of the present invention, there is provided a pharmaceutical composition comprising two or more antisense oligonucleotides complementary to a thymidylate synthase mRNA or analogues of said oligonucleotides and a pharmaceutically acceptable carrier.

In accordance with another aspect of the present invention, there is provided a use of a composition comprising two or more antisense oligonucleotides complementary to a thymidylate synthase mRNA or analogues of said oligonucleotides in the treatment of cancer.

In accordance with another aspect of the present invention, there is provided a use of a composition comprising two or more antisense oligonucleotides complementary to a thymidylate synthase mRNA or analogues of said oligonucleotides in conjunction with one or more chemotherapeutic agent in the treatment of cancer.

In accordance with another aspect of the present invention, there is provided a use of a composition comprising two or more antisense oligonucleotides complementary to a thymidylate synthase mRNA or analogues of said oligonucleotides to sensitise neoplastic cells to a chemotherapeutic agent.

In accordance with another aspect of the present invention, there is provided a combination of two or more antisense oligonucleotides complementary to a thymidylate synthase mRNA for use in the treatment of cancer together with one or more chemotherapeutic agents, wherein the use of the combination enhances the anti-tumour effect of standard doses of the one or more chemotherapeutic agents.

In accordance with another aspect of the present invention, there is provided a combination of two or more antisense oligonucleotides complementary to different regions of a thymidylate synthase mRNA for use in the treatment of cancer together with a chemotherapeutic agent, wherein the use of the combination reduces the amount of chemotherapeutic required to effectively treat a mammal with cancer.

In accordance with another aspect of the present invention, there is provided a combination of two or more antisense oligonucleotides complementary to different regions of a thymidylate synthase mRNA for use together with a chemotherapeutic agent to treat a mammal, wherein the combination and chemotherapeutic agent reduce the number of neoplastic cells in said mammal.

In accordance with another aspect of the present invention, there is provided a combination of two or more antisense oligonucleotides for use in the treatment of cancer together with one or more chemotherapeutic agents to treat a mammal, wherein the sequences of two oligonucleotides of the combination are selected from the group of SEQ ID NO: 1 and 2, or SEQ ID NO: 1 and 3.

### BRIEF DESCRIPTION OF THE FIGURES

FIG. 1 depicts the quantitation of TS and GAPDH mRNA levels in HeLa cells treated with ODN combinations.

FIG. 2 depicts flow cytometric analysis of cell cycle profiles in HeLa cells treated with ODN combinations.

FIG. 3 depicts the inhibition of proliferation of HeLa cells by treatment with ODN combinations compared to treatment with individual ODNs.

FIG. 4 depicts the sensitivity of HeLa cells to raltitrexed and 5-FUdR following treatment with combinations of TS antisense ODNs.

FIG. 5 depicts the lack of sensitisation of HeLa cells to 5-FUdR by lower concentrations of ODN combinations when compared to ODN 1 alone.

FIG. 6 depicts the enhanced sensitivity of HeLa cells to raltitrexed and 5-FUdR following treatment with varying concentrations of ODN 1.

FIG. 7 depicts the predicted single-stranded regions in TS mRNA.

FIG. 8 provides a schematic representation of ODN structures.

### DETAILED DESCRIPTION OF THE INVENTION

In the present application strategies for the development and design of efficacious combinations of two or more antisense agents that target different regions of a target mWNA molecule are described. The strategies provide for an approach to the rational or empirical design of combination two or more antisense agents effective as cancer therapies. This represents a novel strategy to developing new cancer therapies or improving known cancer therapies with a wide range of applications.

Thus, the invention generally provides for combinations of antisense agents directed against a TS mRNA and other target mRNAs that can be used alone or in combination with one or more chemotherapeutic drug in cancer therapies. The combinations of antisense agents can be used in conjunction with one or more chemotherapeutic to reduce the number of neoplastic cells in a patient compared to the use of the individual antisense agents with the chemotherapeutic(s). In one embodiment of the present invention, selected antisense agent combinations used in conjunction with a chemotherapeutic agent reduce the number of neoplastic cells in a patient to a greater extent than use of the chemotherapeutic alone.

The combinations of the present invention are also useful for enhancing the effects of chemotherapeutics in cancer cells. In one embodiment, the combinations are useful in enhancing the effectiveness of chemotherapeutics in drug-resistant cancers that display elevated levels of TS. The antisense agent combinations of the invention can, therefore, be used to reduce the amount of chemotherapeutic necessary to elicit an effective anti-cancer response.

Definitions

Unless defined otherwise, all technical and scientific terms used herein have the same meaning as commonly understood by one of ordinary skill in the art to which this invention belongs.

The term “antisense oligonucleotide, ” as used herein, means a nucleotide sequence that is complementary to the mRNA of a thymidylate synthase (TS) gene or mRNA of an other “target gene”. In the context of the present invention, the target gene has been implicated in processes that can lead to the development of cancer in a mammal and drug resistant cancers and thus is a suitable target for cancer therapies.

The term “selectively hybridize, ” as used herein, refers to the ability of a nucleic acid to bind detectably and specifically to a second nucleic acid. Polynucleotides, oligonucleotides and fragments thereof selectively hybridize to target nucleic acid strands under hybridization and wash conditions that minimize appreciable amounts of detectable binding to nonspecific nucleic acids. High stringency conditions can be used to achieve selective hybridization conditions as known in the art and discussed herein. Typically, hybridization and washing conditions are performed at high stringency according to conventional hybridization procedures. Washing conditions are typically 1-3×SSC, 0.1-1% SDS, 50-70° C. with achange of wash solution after about 5-30 minutes.

The term “corresponds to,” as used herein, with reference to nucleic acid sequences means a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA”.

The following terms are used herein to describe the sequence relationships between two or more polynucleotides: “reference sequence, ” “comparison window, ” “sequence identity, ” “percentage of sequence identity” and “substantial identity.” A “reference sequence” is a defined sequence used as a basis for a sequence comparison; a reference sequence may be a subset of a larger sequence, for example, as a segment of a full-length mRNA or mRNA sequence, or may comprise a complete mRNA or mRNA sequence, Generally, a reference sequence is at least 20 nucleotides in length, frequently at least 25 nucleotides in length, and often at least 50 nucleotides in length. Since two polynucleotides may each (1) comprise a sequence (i.e. a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e. gaps) of 20 percent or less as compared to the reference sequence (which does not comprise additions or deletions) for optimal alignment of the two sequences. Optimal alignment of sequences for aligning a comparison window may be conducted, for example, by the local homology algorithm of Smith and Waterman, 1981, Adv. Appl. Math. 2:482, by the homology alignment algorithm of Needleman and Wunsch, 1970, J. Mol. Biol. 48:443-453, by the search for similarity method of Pearson and Lipman, 1988, Proc. Natl. Acad. Sci. U.S.A. 85:2444-2448, by computerized implementations of these algorithms (GAP, BESTFIT, FASTA, and TFASTA in the Wisconsin Genetics Software Package Release 7.0, Genetics Computer Group, 573 Science Dr., Madison, Wis. and similar packages), or by inspection, and the best alignment (i.e. resulting in the highest percentage of identity over the comparison window) generated by the various methods is selected.

The term “sequence identity” means that two polynucleotide sequences are identical (i.e. on a nucleotide-by-nucleotide basis) over the comparison window. The term “percentage of sequence identity” is calculated by comparing two optimally aligned sequences over the comparison window, determining the number of positions at which the identical nucleic acid base (e.g. A, T, C, G, U, or I) occurs in both sequences to yield the number of matched positions, dividing the number of matched positions by the total number of positions in the comparison window (i.e. the window size), and multiplying the result by 100 to yield the percentage of sequence identity.

The term “substantial identity, ” as used herein, denotes a characteristic of a polynucleotide sequence, wherein the polynucleotide comprises a sequence that has at least 30 percent sequence identity, often at least 50 percent sequence identity, and more usually at least 60 percent sequence identity as compared to a reference sequence over a comparison window of at least 20 nucleotide positions, and frequently over a window of at least 25-50 nucleotides, wherein the percentage of sequence identity is calculated by comparing the reference sequence to the polynucleotide sequence which may include deletions or additions which total 20 percent or less of the reference sequence over the comparison window.

Antisense Molecules of the Present Invention

Selection and Characteristics

“Targeting” an antisense compound to a thymidylate synthase mRNA or another “target gene” mRNA, in the context of the present invention, is a multistep process. The process usually begins with the identification of a target nucleic acid sequence whose function is to be modulated. For the present invention, the target is the cellular gene (or mRNA transcribed from the gene) encoding TS. Sequences for various TS mRNAs are known in the art, for example, X02308, NM_{—}019179, NM_{—}001071, XM_{—}131960, AB077208, AB077207, L12138, and M30774. The targeting process also includes determination of a site or sites within this gene for the antisense interaction to occur such that the desired effect, e.g. detection or modulation of expression of the protein encoded by the gene, will result. Once the target site or sites have been identified, oligonucleotides are chosen that are sufficiently complementary (i.e. hybridize with sufficient strength and specificity) to the target mRNA to give the desired result.

Generally, there are five regions of a mRNA that may be targeted for antisense modulation: the 5′ untranslated region (5′-UTR), the translation initiation or start codon region, the open reading frame (ORF), the translation termination or stop codon region and the 3′ untranslated region (3′-UTR). Regions of an mRNA may be targeted, wherein known regulatory sequence elements have been identified (e.g. for post-transcriptional control and mRNA stability), or that are unique, for example, to a group of mRNAs encoding for similar proteins.

As is known in the art, the terms “translation initiation codon” and “start codon” can encompass many codon sequences, even though the initiator amino acid in eukaryotes is typically methionine and that eukaryotic genes may have two or more alternative start codons, any one of which may be preferentially utilised for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding TS regardless of the sequence(s) of such codons.

It is also known in the art that some eukaryotic transcripts are directly translated, however, most mammalian open reading frames (ORFs) contain one or more sequences, known as “introns,” which are excised from a transcript before it is translated; the expressed (unexcised) portions of the ORF are referred to as “exons” (Alberts et al., 1983, Molecular Biology of the Cell, Garland Publishing Inc., New York, pp. 411-415). In the context of the present invention, both introns and exons may serve as targets for antisense.

In some instances, an ORF may also contain one or more sites that may be targeted for antisense modulation due to some functional significance in vivo. Examples of the latter types of sites include intragenic stem-loop structures (see, for example, U.S. Pat. No. 5,512,438) and, in unprocessed mRNA molecules, intron/exon splice sites. In addition, mRNA molecules possess a 5′ cap region that may also serve as a target for antisense modulation. The 5′ cap of an mRNA comprises an N⁷-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap.

There are also elements in the 3′-UTR region which can impact upon message stability, including examples of unique cis-elements that interact with trans-acting proteins to control mRNA turnover rates (Hal(e and Richer, 1997, Biochim. Biophys. Acta 1332: M31-M38). In addition, the polyadenylated tail can serve several functions impacting upon translation efficiency and message turnover, for example, by protecting the message from degradation, depending on the length of the poly ‘A’ tail (Ford et al., 1997, Mol. Cell. Biol. 17:398-406).

Thus, the antisense oligonucleotides according to the present invention can be complementary to regions of the complete target gene including the introns, or the antisense oligonucleotides can be complementary to part of the mRNA region from the target gene.

In accordance with the invention, suitable combinations of antisense ODNs to TS include, but are not limited to, those in which at least one of the antisense ODNs targets the 3′-UTR region of a TS mRNA; those comprising antisense ODNs that target the 3′-UTR region and the coding region of a TS mRNA; those comprising antisense ODNs that target the 3′-UTR and 5′-UTR of the TS mRNA; those comprising antisense ODNs that target the 3′-UTR and the region spanning the stop codon of the TS mRNA; those comprising antisense ODNs that target the 3′-UTR and the region spanning the start codon of the TS mRNA; those comprising antisense ODNs that target the regions spanning the start and stop codon of the TS mRNA; those comprising antisense ODNs that target the 5′-UTR and the region spanning the stop codon of the TS mRNA; those comprising antisense ODNs that target the 5′-UTR region of the TS mRNA; those comprising antisense ODNs that target the 5′-UTR and the coding regions of the TS mRNA; and combinations thereof.

The antisense agents in accordance with the present invention are selected from a sequence complementary to the target gene such that the sequence exhibits the least likelihood of forming duplexes, hair-pins, or of containing homooligomer or sequence repeats. The oligonucleotide may further contain a GC clamp. One skilled in the art will appreciate that these properties can be determined qualitatively using various computer modelling programs, for example, the program OLIGO® Primer Analysis Software, Version 5.0 (distributed by National Biosciences, Inc., Plymouth, Minn.).

Alternatively, or in addition, the antisense oligonucleotides can be selected on the basis that the sequence is highly conserved for the target gene between two or more species. These properties can be determined using the BLASTN program (Altschul, et al., 1990, J. Mol. Biol., 215:403-10) of the University of Wisconsin Computer group (GCG) software (Devereux et al., 1984, Nucleic Acids Res., 12:387-395) with the National Center for Biotechnology Information (NCBI) databases.

In order to be effective, antisense oligonucleotides are typically between 7 and 100 nucleotides in length. In one embodiment of the present invention the antisense oligonucleotides comprise from at least about 7 to about 50 nucleotides, or nucleotide analogues. In a related embodiment the antisense oligonucleotides comprise from about 12 to about 35 nucleotides, or nucleotide analogues, and in another embodiment from about 15 to about 25 nucleotides, or nucleotide analogues.

It is understood in the art that an antisense oligonucleotide need not have 100% identity with the complement of its target sequence. The antisense oligonucleotides in accordance with the present invention have a sequence that is at least about 75% identical to the complement of the target sequence. In one embodiment of the present invention, the antisense oligonucleotides have a sequence that is at least about 90% identical to the complement of the target sequence. In a related embodiment, they have a sequence that is at least about 95% identical to the complement of the target sequence, allowing for gaps or mismatches of several bases. Identity can be determined, for example, by using the BLASTN program of the University of Wisconsin Computer Group (GCG) software.

In order for the antisense oligonucleotides of the present invention to function in inhibiting expression of the target mRNA, it is necessary that they demonstrate adequate specificity for the target sequence and do not bind to other non-TS sequences in the cell. Therefore, in addition to possessing an appropriate level of sequence identity to the complement of the target sequence, the antisense oligonucleotides of the present invention should not closely resemble other known sequences. The antisense ODNs of the present invention, therefore, have less than 15 nucleotides identical to any other non-TS sequence. In one embodiment, the antisense ODNs have less than 12 nucleotides identical to any other non-TS sequence. In another embodiment, the antisense ODNs have less than 7 nucleotides identical to any other non-TS sequence.

It will, however, be appreciated by one skilled in the art that the degree of acceptable identity between sequences may vary, for example, according to the length of the antisense oligonucleotides and the relative position of the identical nucleotides in the sequences within a comparison window, such that greater than a 15 nucleotide identity may exist, and the antisense ODN still demonstrates adequate specificity for a target sequence. The identity of the antisense oligonucleotides of the present invention to other sequences can be determined, for example, through the use of the BLASTN program and the NCBI databases as indicated above.

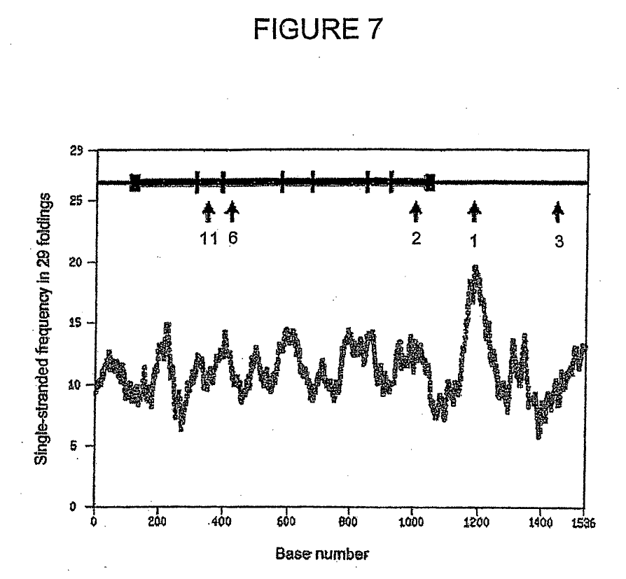

As is known in the art, mRNA molecules frequently comprise regions of secondary structure and mRNA folding may interfere with the ability of an antisense molecule to bind to its target region. Regions of secondary structure in an mRNA molecule can be predicted using known algorithms (for example, Mathews et al., 1999, J Mol Biol. 288:911-940; Zuker et al., In: Barciszewski J, Clark B F C, eds. RNA Biochemistry and Biotechnotogy. New York, N.Y.: Kluwer Academic Publishers; 1999, 11-43). Predicted single-stranded regions for the human TS mRNA are shown in FIG. 7. In one embodiment of the present invention, the antisense ODNs for use in the combinations are targeted to regions of the TS mRNA that are predicted to be single-stranded.

In one embodiment of the present invention, one or more of the antisense ODNs or other antisense agents for use in the combinations comprise at least 5 consecutive nucleotides or nucleotide base sequences from one of the sequences provided in Table 1. In another embodiment, one or more of the antisense ODNs or other antisense agents for use in the combinations comprise at least 7 consecutive nucleotides or nucleotide base sequences from one of the sequences provided in Table 1. In another embodiment, one or more of the antisense ODNs or other antisense agents for use in the combinations comprise at least 10 consecutive nucleotides or nucleotide base sequences from one of the sequences provided in Table 1.

TABLE 1

Exemplary Antisense ODNs against Human TS

| | | |
|---|---|---|
| | Complementary | |
| | Region in TS mRNA | |
| | (based on | |
| | GenBank ™ Accession | SEQ ID |
| Sequence (5′→3′) | No. NM_001071) | NO |

| | | |
|---|---|---|
| GCCAGTGGCAACATCCTTAA | 1184-1203 | 1 |
| TTGGATGCGGATTGTACCCT | 1002-1021 | 2 |
| ACTCAGCTCCCTCAGATTTG | 1436-1455 | 3 |
| CCAGCCCAACCCCTAAAGAC | 1081-1100 | 5 |
| GGCATCCCAGATTTTCACTC | 419-438 | 6 |
| AGCATTTGTGGATCCCTTGA | 380-399 | 11 |
| GGCCGGCGCGGCAGCTCCGA | 121-140 | 13 |
| GCCGGCCACAGGCATGGCGC | 101-120 | 14 |
| GCAACTCCTCCAAAACACCC | 351-370 | 15 |
| AGCATTTGTGGATCCCTTGA | 380-399 | 16 |
| GGCCCATGTCTCCCGATCTC | 747-766 | 17 |
| CAGGCCCGTGATGTGCGCAA | 815-834 | 18 |
| AAAGGTCTGGGTTCTCGCTG | 1003-1022 | 19 |

It will be appreciated that the invention is not restricted merely to those specific antisense oligonucleotides which are disclosed in Table 1 above but encompasses oligonucleotides of from about 5 to about 100 nucleotides in length, for example from about 7 to about 20 nucleotides in length, which selectively inhibit thymidylate synthase production and which are selected from those regions of the TS gene which are described hereinbefore.

The term “antisense agents” as used herein includes oligomeric antisense compounds, including oligonucleotide mimetics, modified oligonucleotides, and chimeric antisense compounds. Chimeric antisense compounds are antisense compounds that contain two or more chemically distinct regions, each made up of at least one monomer unit.

Modifications to Antisense Oligonucleotides

In the context of this invention, the term “oligonucleotide” refers to an oligomer or polymer of ribonucleic acid (RNA), deoxyribonucleic acid (DNA), or modified versions thereof, or RNA or DNA mimetics. This term, therefore, includes oligonucleotides composed of naturally-occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for the nucleic acid target and increased stability in the presence of nucleases. The oligonucleotides according to the present invention can be single-stranded or they can be double-stranded.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of antisense agents useful in this invention include those containing modified backbones or non-natural internucleoside linkages. As defined in this specification, antisense oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary modified oligonucleotide backbones that can be incorporated into the oligonucleotides according to the present invention include, for example, phosphorothioates, chiral phosphorothioates, bridged phosphorothioates, phosphorodithioates, phosphotriesters, aminoalkylphosphotriesters, methyl and other alkyl phosphonates including 3′-alkylene phosphonates, bridged methylene phosphonates and chiral phosphonates, phosphinates, phosphoramidates including 3′-amino phosphoramidate, bridged phosphoramidates and aminoalkylphosphoramidates, thionophosphoramidates, thionoalkylphosphonates, thionoalkylphosphotriesters, and boranophosphates having normal 3′-5′ linkages, 2′-5′ linked analogues of these, and analogues having inverted polarity wherein the adjacent pairs of nucleoside units are linked 3′-540 to 5′-3′ or 2′-5′ to 5′-2′. Various salts, mixed salts and free acid forms are also included.

In one embodiment of the present invention, one or more of the antisense agents of the combination comprise at least one phosphorothioate linkage. In another embodiment, one or more of the antisense agents comprise phosphorothioate internucleotide linkages that link the four, five or six 3′-terminal nucleotides of the oligonucleotide. In a further embodiment, one or more of the antisense agents comprise phosphorothioate internucleotide linkages that link all the nucleotides of the oligonucleotide.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulphide, sulphoxide and sulphone backbones; formacetyl and thioformacetyl backbones; methylene formacetyl and thioformacetyl backbones; carbonate backbones; carboxymethylester backbones; acetamidate backbones; carbamate backbones; thioether backbones; alkene containing backbones; sulphamate backbones; methyleneimino and methylenehydrazino backbones; sulphonate and sulphonamide backbones; amide backbones; and others having mixed N, O, S and CH₂ component parts.

The term “alkyl” as used herein refers to monovalent alkyl groups having from 1 to 20 carbon atoms. In one embodiment of the present invention the alkyl group has between 1 and 6 carbon atoms. Examples of suitable alkyl groups include, but are not limited to, methyl, ethyl, n-propyl, iso-propyl, n-butyl, iso-butyl, n-hexyl, and the like.

The term “cycloalkyl” refers to cyclic alkyl groups of from 3 to 20 carbon atoms having a single cyclic ring or multiple condensed rings. Examples of suitable cycloalkyl groups include, but are not limited to, single ring structures such as cyclopropyl, cyclobutyl, cyclopentyl, cyclooctyl, and the like, or multiple ring structures such as adamantanyl, and the like.

The present invention also contemplates oligonucleotide mimetics in which both the sugar and the internucleoside linkage of the nucleotide units are replaced with novel groups. The base units are maintained for hybridization with an appropriate nucleic acid target. An example of such an oligonucleotide mimetic, which has been shown to have excellent hybridization properties, is a peptide nucleic acid (PNA) [Nielsen et al., Science, 254:1497-1500 (1991)]. In PNA compounds, the sugar-backbone of an oligonucleotide is replaced with an amide-containing backbone, in particular an aminoethylglycine backbone. The nucleobases are retained and are bond directly or indirectly to aza-nitrogen atoms of the amide portion of the backbone.

The present invention also contemplates one or more antisense agents that are oligonucleotides comprising “locked nucleic acids” (LNAs), which are novel conformationally restricted oligonucleotide analogues containing a methylene bridge that connects the 2′-O of ribose with the 4′-C (see, Singh et al, Chem. Commun., 1998, 4:455-456). LNA oligonucleotides contain one or more nucleotide building blocks in which an extra methylene bridge, as noted above, that fixes the ribose moiety either in the C3′-endo (β-D-LNA) or C2′-endo (α-L-LNA) conformation.

LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mismatched sequences show that LNA obey the Watson-Crick base pairing rules with generally improved selectivity compared to the corresponding unmodified reference strands. Antisense oligonucleotides containing LNAs have been described (Wahlestedt et al., Proc. Natl. Acad. Sci. U.S.A., 2000, 97:5633-5638), which were efficacious and non-toxic. In addition, the LNA/DNA copolymers were not degraded readily in blood serum and cell extracts.

LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

The one or more antisense agents comprising LNAs can be designed as “gapmers” in which the oligonucleotide comprises a stretch of LNAs at the 5′ end, followed by a “gap” of DNA nucleotides, then a second stretch of LNAs at the 3′ end.

In one embodiment, one or more of the antisense agents of the invention comprise LNAs. In another embodiment, one or more of the antisense agents of the invention comprise β-D-LNAs. In a further embodiment, one or more of the antisense agents of the invention are LNA gapmers, as described above.

Modified oligonucleotides according to the present invention may also contain one or more substituted sugar moieties. For example, oligonucleotides may comprise sugars with one of the following substituents at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoallkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂CH₂OCH₃, also known as 2′-O-(2-methoxyethyl) or 2′-MOE) [Martin el al., Helv. Chim. Aecta, 78:486-504(1995)], 2′-dimethylaminooxyethoxy (O(CH₂)₂ON(CH₃)₂ group, also known as 2′-DMAOE), 2′-methoxy (2-O—CH₃), 2-aminopropoxy (2-OCH₂CH₂CH₂NH₂) and 2-fluoro (2-F).

In one embodiment of the present invention, one or more of the antisense agents comprise at least one nucleotide comprising a substituted sugar moiety. In another embodiment, one or more of the antisense agents comprises at least one 2′-O-(2-methoxyethyl) or 2′-MOE modified nucleotide.

Similar modifications may also be made at other positions on the oligonucleotide, particularly the 3′ position of the sugar on the 3′ terminal nucleotide or in 2′-5′ linked oligonucleotides and the 5′ position of the 5′ terminal nucleotide. Oligonucleotides may also have sugar mimetics such as cyclobutyl moieties in place of the pentofuranosyl sugar.

Oligonucleotides according to the present invention may also include modifications or substitutions to the nucleobase. As used herein, “′ unmodified” or “natural” nucleobases include the purine bases adenine (A) and guanine (G), and the pyrinudine bases thymine (T), cytosine (C) and uracil (U). Modified nucleobases include other synthetic and natural nucleobases such as 5-methylcytosine (5-me-C); inosine; 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioallkyl, 8-hydroxyl and other 8-substituted adenines and guanines; 5-halo particularly 5-bromo, 5-trifluoromethyl and other 5-substituted uracils and cytosines; 7-methylguanine and 7-methyladenine; 8-azaguanine and 8-azaadenine; 7-deazaguanine and 7-deazaadenine; 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

Another oligonucleotide modification included in the present invention is the chemical linkage to the oligonucleotide of one or more moieties or conjugates which enhance the activity, cellular distribution or cellular uptake of the oligonucleotide. Such moieties include, but are not limited to, lipophilic or lipid moieties such as a cholesterol moiety [Letsinger et al., Proc. Anatl. Acad. Sci. USA, 86:6553-6556 (1989)], cholic acid [Manoharan et al., Bioorg. Med. Chem. Let., 4:1053-1060 (1994)], a thioether, e.g. hexyl-S-tritylthiol [Manoharan et al., Ann. N.Y. Acad. Sci., 660:306-309 (1992); Manoharan et al., Bioorg. Med. Chem. Lett., 3:2765-2770 (1993)], a thiocholesterol [Oberhauser et al., Nucl. Acids Res., 20:533-538 (1992)], an aliphatic chain, e.g. dodecandiol or undecyl residues [Saison-Behmoaras et al., EMBO J, 10:1111-1118 (1991); Kabanov et al., FEBS Lett., 259:327-330 (1990); Svinarchuk et al., Biochimie, 75:49-54 (1993)], a phospholipid, e.g. di-hexadecyl-rac-glycerol or triethylammonium 1,2-di-O-hexadecyl-rac-glycero-3-H-phosphonate [Manoharan et al., Tetrahedron Lett., 36:3651-3654 (1995); Shea et al., Nucl. Acids Res., 18:3777-3783 (1990)], a polyamine or a polyethylene glycol chain [Manoharanet al., Nucleosides & Nucleotides, 14:969-973 (1995)], or adamantane acetic acid [Manoharan et al., Tetrahedron Lett., 36:3651-3654 (1995)], a palmityl moiety [Mishra el al., Biochim. Biophys. Acta, 1264:229-237 (1995)], or an octadecylamine or hexylamino-carbonyl-oxycholesterol moiety [Crooke et al., J. Pharmacol. Exp. Ther., 277:923-937 (1996)].

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide.

The present invention thus further includes one or more antisense agents that are chimeric compounds. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease that cleaves the RNA strand of an RFNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridising to the same target region. Cleavage of the RNA target can be routinely detected by gel electrophoresis and, if necessary, associated nucleic acid hybridisation techniques known in the art.

In one embodiment of the present invention, one or more of the antisense agents comprise a phosphorothioate backbone in combination with at least one 2′-MOE modified sugar. In another embodiment, one or more of the antisense agents comprise a phosphorothioate backbone in combination with one or more 2′-MOE modified sugars at the 3′ and 5′ ends of the oligonucleotide. In a further embodiment, one or more of the antisense agents are “methoxy-ethoxy winged” oligonucleotides with one or more 2′-MOE modified sugars at the 3′ and 5′ ends of the oligonucleotide.

In the context of the present invention, an antisense oligonucleotide is “nuclease resistant” when it has either been modified such that it is less susceptible to degradation by DNA and/or RNA nucleases than when in its non-modified form or, alternatively, has been placed in a delivery vehicle which itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes. In one embodiment of the present invention, one or more of the antisense agents are nuclease resistant.

The present invention further contemplates antisense oligonucleotides that contain groups for improving the pharmacokinetic properties of the oligonucleotide, or groups for improving the pharmacodynamic properties of the oligonucleotide.

In one embodiment of the present invention, the one or more of the antisense agents comprise at least one phosphorothioate linkage. In another embodiment, one or more of the antisense agents comprise at least one 2′-methoxy-ethoxy substituted nucleotide. In another embodiment, one or more of the antisense agents comprise a plurality of 2′-methoxy-ethoxy substituted nucleotides at both the 3′- and 5′-end of the oligonucleotide. In still another embodiment, one or more of the antisense agents comprise both at least one phosphorothioate linkage and a plurality of 2′-methoxy-ethoxy substituted nucleotides at both the 3′- and 5′-ends of the oligonucleotide.

Short Interfering RNA (siRNA) Molecules

The present invention further contemplates that one or more of the antisense agents may be in the form of siRNA molecules. RNA interference mediated by double-stranded siRNA molecules, which are generated in nature when long double-stranded RNA molecules are cleaved by the action of an endogenous ribonuclease, is known in the art to play an important role in post-transcriptional gene silencing [Zamore, Nature Struc. Biol., 8:746-750 (2001)]. Transfection of mammalian cells with synthetic siRNA molecules having a sequence identical to a target gene has been demonstrated to result in a reduction in the mRNA levels of the target gene [see, for example, Elbashir, et al., Nature, 411:494-498 (2001)]. siRNA molecules are typically 21-22 base pairs in length.

The specificity of siRNA molecules is determined by the binding of the antisense strand of the molecule to its target mRNA. Thus, one or more of the antisense agents of the present invention can be provided as part of an siRNA molecule which is targeted to a specific gene. As is known in the art, effective siRNA molecules should be less than 30 to 35 base pairs in length to prevent them triggering non-specific RNA interference pathways in the cell via the interferon response. Thus, in one embodiment of the present invention, one or more of the antisense agents are siRNA molecules between about 15 and about 25 base pairs in length. In a related embodiment, they are between 19 and 22 base pairs in length.

The double-stranded siRNA molecules can further comprise poly-T or poly-U overhangs at each end to minimise RNase-mediated degradation of the molecules. In another embodiment of the present invention, one or more of the antisense agents are siRNA molecules comprising overhangs at the 3′ and 5′ ends which consist of two thymidine or two uridine residues. Design and construction of siRNA molecules is known in the art [see, for example, Elbashir, et al., Nature, 411:494-498 (2001); Bitko and Barik, BMC Microbiol., 1:34 (2001)]. In addition, kits that provide a rapid and efficient means of constructing siRNA molecules by in vitro transcription are also commercially available (Ambion, Austin, Tex.; New England Biolabs, Beverly, Mass.).

Single-stranded siRNA and short-hairpin siRNA (shRNA) molecules are also known in the art. The present invention contemplates that one or more of the antisense agents can be provided as single-stranded siRNTA molecules and/or as shRNA molecules,

siRNA molecules can comprise naturally-occurring nucleobases, sugars and covalent internucleoside (backbone) linkages as well as oligonucleotides having non-naturally-occurring portions, which function similarly, as described above.

Design of'siRNA Molecules

Following selection of an appropriate target mRNA sequence, siRNA molecules that comprise a nucleotide sequence complementary to all or a portion of the target sequence, i.e. an antisense sequence, can be designed and prepared. As indicated above, the siRNA molecule can be double stranded (i. e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

The siRNA molecule can be a double-stranded RNA (dsRNA) comprising two separate complementary RNA strands. The RNA strands of the dsRNA may be the same length in nucleotides, or may be different in length. In one embodiment, one or more of the antisense agents is an siRNA which is a dsRNA. In another embodiment, the siRNA is a dsRNA wherein both RNA strands are the same length.

In one embodiment, one or more of the antisense agents are dsRNA molecules and also include siRNA molecules assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi.

Small hairpin RNA (shRNA) molecules thus are also contemplated by the present invention as one or more antisense agents. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form, a dsRNA molecule (optionally with additional processing steps that may result in addition or removal of one, two, three or more nucleotides from the 3′ end and/or the 5′ end of either or both strands). The spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). The spacer sequence is typically an unrelated nucleotide sequence that is situated between two complementary nucleotide sequence regions which, when annealed into a double-stranded nucleic acid, comprise a shRNA (see, for example, Brummelkamp et al., 2002 Science 296:550; Paddison et al., 2002 Genes Develop. 16:948; Paul et al, Nat. Biotechnol. 20:505-508 (2002); Grabarek et al., BioTechniques 34:734-44 (2003)). The spacer sequence generally comprises between about 3 and about 100 nucleotides.

The ssRNA molecules as one or more antisense agents according to the present invention are generally single-stranded RNA molecules with little or no secondary structure.

The overall length of the one or more antisense agents which comprise siRNA molecules of the present invention can vary from about 14 to about 200 nucleotides depending on the type of siRNA molecule being designed. Between about 14 and about 50 of these nucleotides are complementary to the mRNA target sequence, i.e. constitute the specific antisense sequence of the siRNA molecule. For example, when the siRNA molecule is a dsRNA or ssRNA molecule, the length can vary from about 14 to about 50 nucleotides, whereas when the siRNA is a shRNA or circular molecule, the length can vary from about 40 nucleotides to about 200 nucleotides.

In one embodiment of the present invention, one or more of the antisense agents are a dsRNA or ssRNA molecule between about 15 and about 40 nucleotides in length. In another embodiment, the siRNA molecule is a dsRNA or ssRNA molecule between about 15 and about 35 nucleotides in length. In another embodiment, the siRNA molecule is a dsRNA or ssRNA molecule between about 17 and about 30 nucleotides in length. In another embodiment, the siRNA molecule is a dsRNA or ssRNA molecule between about 19 and about 25 nucleotides in length. In another embodiment, the siRNA molecule is a dsRNA or ssRNA molecule between about 21 to about 23 nucleotides in length.

In an alternative embodiment, one or more of the antisense agents are a shRNA molecule or circular siRNA molecule between about 50 and about 100 nucleotides in length. In a further embodiment, the siRNA molecule is a shRNA molecule between about 50 to about 60 nucleotides in length.

As indicated above, the siRNA molecule comprises an antisense strand that includes a specific antisense sequence complementary to all or a portion of a target mRNA sequence. One skilled in the art will appreciate that the entire length of the antisense strand comprised by the siRNA molecule does not need to be complementary to the target sequence. Thus, the antisense strand of the siRNA molecules may comprise a specific antisense sequence together with nucleotide sequences at the 5′ and/or 3′ termini that are not complementary to the target sequence. Such non-complementary nucleotides may provide additional functionality to the siRNA molecule. For example, they may provide a restriction enzyme recognition sequence or a “tag” that facilitates detection, isolation or purification. Alternatively, the additional nucleotides may provide a self-complementary sequence that allows the siRNA to adopt a hairpin configuration. Such configurations are useful when the siRNA molecule is a shRNA molecule, as described above.

Accordingly, within its overall length of about 14 to about 200 nucleotides, the one or more antisense agents that are siRNA molecules comprise a specific antisense sequence of between about 14 to about 50 nucleotides in length that is complementary to all or a portion of a selected target mRNA sequence. In one embodiment, the length of the specitic antisense sequence is from about 14 to about 40 nucleotides. In another embodiment, the length of the specific antisense sequence is from about 14 to about 35 nucleotides. In another embodiment, the length of the specific antisense sequence is from about 14 to about 30 nucleotides. In another embodiment, the length of the specific antisense sequence is from about 15 to about 30 nucleotides. In a further embodiment, the length of the specific antisense sequence is from about 17 to about 30 nucleotides. In other embodiments, the length of the specific antisense sequence is from about 19 to about 25, from about 19 to about 23, and from about 21 to about 23 nucleotides.

The specific antisense sequence comprised by the siRNA molecule can be identical or substantially identical to the complement of the target sequence. In one embodiment of the present invention, the specific antisense sequence comprised by the siRNA molecule is at least about 75% identical to the complement of the target mRNA sequence. In another embodiment, the specific antisense sequence comprised by the siRNA molecule is at least about 90% identical to the complement of the target mRNA sequence. In a further embodiment, the specific antisense sequence comprised by the siRNA molecule is at least about 95% identical to the complement of the target mRNA sequence. In another embodiment, the specific antisense sequence comprised by the siRNA molecule is at least about 98% identical to the complement of the target mRNA sequence. Methods of determining sequence identity are known in the art and can be determined, for example, by using the BLASTN program of the University of Wisconsin Computer Group (GCG) software or provided on the NCBI website.

The specific antisense sequence of the one or more antisense agents which comprise at least one siRNA molecule may exhibit variability by differing (e.g. by nucleotide substitution, including transition or transversion) at one, two, three, four or more nucleotides from the sequence of the target mRNA. When such nucleotide substitutions are present in the antisense strand of a dsRNA molecule, the complementary nucleotide in the sense strand with which the substitute nucleotide would typically form hydrogen bond base-pairing may or may not be correspondingly substituted. dsRNA molecules in which one or more nucleotide substitution occurs in the sense sequence, but not in the antisense strand, are also contemplated by the present invention. When the antisense sequence of an siRNA molecule comprises one or more mismatches between the nucleotide sequence of the siRNA and the target nucleotide sequence, as described above, the mismatches may be found at the 3′ terminus, the 5′ terminus or in the central portion of the antisense sequence.

In another embodiment of the invention, the one or more antisense agents which comprise at least one siRNA molecule comprise a specific antisense sequence that is capable of selectively hybridizing under stringent conditions to a portion of a naturally occurring target gene or target mRNA. Suitable stringent conditions include, for example, hybridization according to conventional hybridization procedures and washing conditions of 1-3×SSC, 0.1-1% SDS, 50-70° C. with a change of wash solution after about 5-30 minutes. As known to those of ordinary skill in the art, variations in stringency of hybridization conditions may be achieved by altering the time, temperature, and/or concentration of the solutions used for the hybridization and wash steps. Suitable conditions can also depend in part on the particular nucleotide sequences used, for example the sequence of the target mRNA or gene.

siRNA molecules having a duplex or double-stranded structure, for example dsRNA or shRNA, can have blunt ends, or can have 3′ and/or 5′ overhangs. As used herein, “overhang” refers to the unpaired nucleotide or nucleotides that protrude from a duplex structure when a 3′-terminus of one RNA strand extends beyond the 5′-terminus of the other strand (3′ overhang), or vice versa (5′ overhang). The nucleotides comprising the overhang can be ribonucleotides, deoxyribonucleotides or modified versions thereof. In one embodiment, at least one strand of the siRNA molecule has a 3′ overhang from about 1 to about 6 nucleotides in length. In other embodiments, the 3′ overhang is from about 1 to about 5 nucleotides, from about 1 to about 3 nucleotides and from about 2 to about 4 nucleotides in length.

When the siRNA molecule comprises a 3′ overhang at one end of the molecule, the other end can be blunt-ended or have also an overhang (5′ or 3′). When the siRNA molecule comprises an overhang at both ends of the molecule, the length of the overhangs may be the same or different. In one embodiment, the siRNA molecule comprises 3′ overhangs of about 1 to about 3 nucleotides on both ends of the molecule. In a further embodiment, the siRNA molecule is a dsRNA having a 3′ overhang of 2 nucleotides at both ends of the molecule. In yet another embodiment, the nucleotides comprising the overhang of the siRNA are TT dinucleotides or UU dinucleotides.

When determining the percentage identity of the siRNA molecule comprising one or more overhangs to the target mRNA sequence, the overhang(s) may or may not be taken into account. For example, the nucleotides from a 3′ overhang and up to 2 nucleotides from the 5′- and/or 3′-terminus of the double strand may be modified without significant loss of activity of the siRNA molecule.

In the context of the invention, the term “RNA” refers to a nucleic acid molecule of one or more ribonucleotides in length, wherein the ribonucleotide(s) are naturally-occurring ribonucleotides or modified ribonucleotides. In general RNA refers to an oligomer or polymer of ribonucleotides. The siRNA molecules, therefore, can be composed of naturally-occurring nucleobases, sugars and covalent internucleoside (backbone) linkages or can comprise one or more non-naturally-occurring nucleotides or linkages, which function similarly. Such modified siRNA molecules are-often preferred over native forms because of desirable properties such as, for example, enhanced cellular uptake and/or increased bioavailability, enhanced affinity for nucleic acid target, and increased stability in the presence of nucleases. For example, certain chemical modifications can improve the bioavailability of the siRNA molecules by targeting particular cells or tissues and/or improving cellular uptake of the molecule. Typically, siRNA molecules comprising modified nucleotides and/or linkages retain substantially the same activity as the unmodified molecule. However, it is also contemplated that the activity of a modified siRNA molecule may be reduced as compared to an unmodified siRNA molecule, but the overall activity of the modified siRNA molecule can be greater than that of the unmodified siRNA molecule due to improved stability and/or delivery of the molecule. Modified siRNA molecules can also minimise the possibility of activating interferon activity.

Preparation of the Antisense Oligonucleotides

The antisense oligonucleotides of the present invention can be synthesized by conventional techniques well-known to those skilled in the art (see, for example, U.S. Pat. No. 6,087,489). For example, the oligonucleotides can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods.

The isolation and purification of antisense oligonucleotides can be accomplished using, for example, filtration, extraction, crystallization, different forms of chromatography, including column, thin layer, preparative low or high pressure liquid chromatography, or a combination of these procedures, in addition to other equivalent separation or isolation procedures.

Alternatively, the antisense oligonucleotides of the present invention can be prepared by enzymatic digestion of the naturally occurring target gene by methods known in the art.

Antisense oligonucleotides can also be prepared through the use of recombinant methods. The present invention, therefore, encompasses expression vectors comprising nucleic acid sequences that encode the antisense oligonucleotides and expression of the encoded antisense oligonucleotides in a suitable host cell. Such expression vectors can be readily constructed using procedures known in the art. Examples of suitable vectors include, but are not limited to, plasmids, phagemids, cosmids, bacteriophages, baculoviruses and retroviruses, and DNA viruses. One skilled in the art will understand that selection of the appropriate host cell for expression of the antisense oligonucleotide will be dependent upon the vector chosen. Examples of host cells include, but are not limited to, bacterial, yeast, insect, plant and mammalian cells.

One skilled in the art will also understand that the expression vector may further include regulatory elements, such as transcriptional elements, required for efficient transcription of the antisense oligonucleotide sequences. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding an antisense oligonucleotide. One skilled in the art will appreciate that selection of suitable regulatory elements is dependent on the host cell chosen for expression of the antisense oligonucleotide and that such regulatory elements may be derived from a variety of sources, including bacterial, fungal, viral, ma malian or insect genes.

In the context of the present invention, the expression vector may additionally contain a reporter gene. Suitable reporter genes include, but are not limited to, β-galactosidase, green fluorescent protein, red fluorescent protein, luciferase, and β-glucuronidase. Incorporation of a reporter gene into the expression vector allows transcription of the antisense oligonucleotide to be monitored by detection of a signal generated by expression of the reporter gene.

In accordance with the present invention, the expression vectors can be introduced into a suitable host cell or tissue by one of a variety of methods known in the art. Such methods can be found generally described in Sambrook et al, 1992, Molecular Cloning: A Laboratory Manual, Cold Spring Harbor Laboratory, New York; Ausubel et al., 1989, Current Protocols in Molecular Biology, John Wiley and Sons, Baltimore Md.; Chang et al., 1995, Somcatic Gene Therapy, CRC Press, Ann Arbor Mich.; Vega et al., 1995, Gene Targeting, CRC Press, Ann Arbor, Mich.; and Vectors: A Survey of Molecular Cloning Vectors and Their Uses, Butterworth's, Boston Mass. (1988) and include, for example, stable or transient transfection, lipofection, electroporation, and infection with recombinant viral vectors.

In Vitro Testing of the Antisense Oligonucleotides

The effectiveness of the antisense oligonucleotides of the present invention in inhibiting target gene expression can be demonstrated initially in vitro, using for example, the method previously described by Choy et al., 1998, Cancer Res. 48: 6949-6952, or the methods described in the Examples.

For example, the antisense ODN can be introduced into a cell line that normally expresses TS or which over-expresses TS (for example, a tumour cell line) and the amount of mRNA transcribed from the TS gene can be measured, for example, by Northern blot analysis. Alternatively, the amount of TS protein produced by the cell can be measured, for example, by Western blot analysis. The amount of mRNA or protein produced in a cell treated with the antisense ODN can then be compared against the amount produced in control cells and will provide an indication of how successfully the antisense ODN has inhibited TS gene expression. Suitable control cells include untreated cells and cells treated with a control, scrambled ODN.

Suitable ODNs for the combinations of the invention are those ODNs which either maintain the levels of TS mRNA and/or TS protein expressed or which decrease these levels. In one embodiment of the invention, at least one antisense ODN for use in a combination decreases the levels of TS mRNA and/or TS protein in a cell.

The colony forming ability or proliferation of neoplastic cells treated with different antisense ODNs or ODN combinations can be tested by growing neoplastic cells to an appropriate density (e.g. approximately 1×10⁴) and then adding an appropriate concentration of one or more antisense ODN in the presence of cationic lipid (e.g. lipofectin to a final concentration of 5 μg/mL). Excess antisense ODN is washed away after a suitable incubation period and the cells are subsequently cultured. Percent inhibition of colony forming ability or proliferation is calculated by comparison of the number of colonies in the treated culture with the number of colonies in control cultures, for example, cultures not pre-treated with antisense ODNs or those pre-treated with a control, scrambled ODN (as visualized, for example, by methylene blue staining).

While a worker skilled in the art will appreciate that the percentage inhibition of colony forming ability or proliferation will be dependent on the particular cell line used, in general, the antisense ODN combinations of the present invention increase the percent inhibition of proliferation of a tumour cell line by at least 5% compared to an individual ODN used alone. In one embodiment, the antisense ODN combinations increase the percent inhibition of proliferation of a tumour cell line by at least 10% compared to an individual ODN used alone. In another embodiment, the antisense ODN combinations increase the percent inhibition of proliferation of a tumour cell line by at least 12% compared to an individual ODN used alone. In other embodiments, the combinations increase the percent inhibition by 12%, 15%, 17%, 20% and 25%.

In Vivo and Ex Vivo Testing of the Antisense Oligonucleotides

The ability of the antisense ODN combinations with or without one or more chemotherapeutics to inhibit tumour growth or proliferation in vivo can be first determined in an appropriate animal model using standard techniques known in the art (see, for example, Enna, et al., Current Protocols in Pharmacology, J. Wiley & Sons, Inc., New York, N.Y.).

In general, current animal models for screening anti-tumour compounds are xenograft models, in which a human tumour has been implanted into an animal. Examples of xenograft models of human cancer include, but are not limited to, human solid tumour xenografts in mice, implanted by sub-cutaneous injection and used in tumour growth assays; human solid tumour isografts in mice, implanted by fat pad injection and used in tumour growth assays; experimental models of lymphoma and leukaemia in mice, used in survival assays; and experimental models of lung metastasis in mice. Examples of currently accepted xenograft models are provided in Table 2.

Antisense oligonucleotides may be administered to neoplastic cells ex vivo prior to injection of the cells into the mice or they may be administered to the mice in vivo after the injection of the cells and tumour establishment in the mice.

When administered in vivo, the antisense oligonucleotides of the present invention can be administered to the animal by, for example, systemic administration (e.g. tail vein injection) or local administration, e.g. into a tumour. Alternatively, the antisense ODNs can be administered by continuous spinal delivery, for example, via an intrathecal catheter attached to a mini-osmotic pump.

As an example, the antisense ODN combinations with one or more chemotherapeutic can be tested in vivo on solid tumours using mice that are subcutaneously grafted bilaterally with a pre-determined amount of a tumour fragment on day 0. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random into groups that will undergo the treatments or act as controls. Suitable groupings would be, for example, those receiving the antisense ODN combination and the one or more chemotherapeutic, those receiving the antisense ODN combination alone, those receiving the chemotherapeutic agent(s) alone and those receiving no treatment or treatment with a control, scrambled ODN. Animals not bearing tumours may also be subjected to the same treatments as the tumour-bearing animals in order to be able to dissociate the toxic effect from the specific effect on the tumour. Chemotherapy generally begins from 3 to 22 days after grafting, depending on the type of tumour, and the animals are observed every day. The combinations of the present invention can be administered to the animals, for example, by bolus infusion. The different animal groups are weighed about 3 or 4 times a week until the maximum weight loss is attained, after which the groups are weighed at least once a week until the end of the trial.

The tumours are measured about 2 or 3 times a week until the tumour reaches a pre-determined size and/or weight, or until the animal dies if this occurs before the tumour reaches the predetermined size/weight. The animals are then sacrificed and the tissue histology, size and/or proliferation of the tumour assessed.

For the study of the effect of the antisense ODN compositions with or without one or more chemotherapeutic on leukaemias, the animals are grafted with a particular number of cells, and the anti-tumour activity is determined by the increase in the survival time of the treated mice relative to the controls.

To study the effect of the antisense ODN combinations with or without one or more chemotherapeutic on tumour metastasis, tumour cells are typically treated ex vivo and then injected into a suitable test animal. The spread of the tumour cells from the site of injection is then monitored over a suitable period of time by standard techniques.

In vivo toxic effects of the oligonucleotides can be evaluated by measuring their effect on animal body weight during treatment and by performing haematological profiles and liver enzyme analysis after the animal has been sacrificed.

TABLE 2
Examples of Xenograft Models of Human Cancer

| Cancer Model | Cell Type |
|---|---|
| Tumour Growth Assay | Prostate (PC-3, DU145) |
| Human solid tumour xenografts | Breast (MDA-MB-231, MVB-9) |
| in mice (sub-cutaneous in- | Colon (HT-29) |
| jection) | Lung (NCI-H460. NCI-H209) |
| | Pancreatic (ASPC-l, 5U86.86) |
| | Pancreatic: drug resistant (BxPC-3) |
| | Skin (A2058, C8161) |
| | Cervical (SIHA, HeLa-S3) |
| | Cervical: drug resistant (HeLa 53-HU- |
| | resistance) |
| | Liver (HepG2) |
| | Brain (U87-MG) |
| | Renal (Caki-1, A498) |
| | Ovary (SK-OV-3) |
| Tumour Growth Assay | Breast: drug resistant (MDA-CDDP-S4, |
| Human solid tumour isografts in | MDA-MB435-To.1) |
| mice (fat pad injection) | |
| Survival Assay | Human: Burkitts lymphoma (Non- |
| Experimental model of | Hodgkin's) (raji) |
| lymphoma and leukaemia in | Murine: erythroleukemia (CB7 Friend |
| mice | retrovirus-induced) |
| Experimental model of lung | Human: melanoma (C8161) |
| metastasis in mice | Murine: fibrosarcoma (R3) |

Applications for the Antisense Oligonucleotides

The antisense ODN combinations of the present invention are useful as drugs for the treatment of cancer or proliferative disorders irrespective of their origin, The antisense ODN combinations may be used alone or in combination with one or more chemotherapeutic. In one embodiment of the present invention, the combinations of antisense ODNs improve the efficacy of the chemotherapeutic and, therefore, can be used to improve standard cancer therapies. This application is particularly important in the treatment of drug-resistant cancers which are not responsive to standard treatment. Drug-resistant cancers can arise, for example, from heterogeneity of tumour cell populations, alterations in response to chemotherapy and increased malignant potential. Such changes are often more pronounced at advanced stages of disease and have, in part, as an underlying cause, changes in genome/message stability. The antisense ODN combinations can also be used to improve the efficacy of chemotherapeutic drugs in the treatment of drug-sensitive tumours and thereby decrease the dosage of the chemotherapeutic(s) required to be administered to a patient. Thus, the antisense ODN combinations of the invention are useful both in improving cancer therapies for drug-resistant tumours by increasing the sensitivity of such tumours to a chemotherapeutic and in reducing the dosages of a chemotherapeutic required for treatment of a drug-sensitive tumour, thereby reducing or minimising side-effects associated with conventional dosages of such drugs.

As is known in the art, TS is frequently overexpressed in a wide variety of neoplastic cells, therefore, the combinations of antisense ODNs against TS mRNA provided by the present invention can be used in the treatment of a broad range of cancers. Examples of cancers which may be treated using the antisense ODN combinations of the invention, include, but are not limited to, carcinomas, leukemias (e.g. of the central-nervous system and blood), lymphomas (Hodgkins and non-Hodgkins), sarcomas, melanomas, adenomas, neuroblastomas, nephroblastomas (e.g. Wilm's tumour) and retinoblastomas.

Examples of carcinomas (i.e. originating in epithelial tissues such as the skin and inner membrane surfaces of the body), include, but are not limited to cancers such as breast cancer, colon cancer, rectal cancer, esophageal cancer, prostate cancer, lung cancer, stomach cancer, bladder cancer, skin cancer, kidney cancer, pancreatic cancer, ovarian cancer, uterine cancer, cervical cancer, cancer of the vulva, liver cancer, thyroid cancer, aveolar cell carcinoma, basal cell carcinoma, bronchogenic carcinoma, chorionic carcinoma, embyronal carcinoma, giant cell carcinoma, glandular carcinoma, medullary carcinoma, melatonic carcinoma, mucinous carcinoma, oat cell carcinoma, scirrhous carcinoma and squamous cell carcinoma.

Examples of sarcomas (i.e. originating in soft tissues of mesenchymal origin such as the connective and supportive tissues of muscle, bone, cartilage and fat), include, but are not limited to cancers such as Kaposi's sarcoma, alveolar soft part sarcoma, bone cancer, botryoid sarcoma, endometrial sarcoma, giant cell sarcoma, osteogenic sarcoma, reticulum cell sarcoma and spindle cell sarcoma, rhabdomyosarcoma and lymphosarcoma.

As indicated above, in the treatment of cancer, the antisense ODN combinations may be used alone or in conjunction with one or more of a variety of chemotherapeutic agents. Chemotherapeutic agents (e.g. synthetic chemical medications) have toxic effects that selectively or non-selectively destroy cancerous tissue. Chemotherapeutics suitable for use with the combinations of the present invention include TS-inhibiting drugs as well as drugs that act through different mechanisms. While there are a variety of chemotherapeutics currently available, many, if not most, have side-effects associated with their use. The search for new chemotherapeutics is, therefore, ongoing and it is likely that new chemotherapeutics will continue to be brought to the market. It will be readily apparent to one skilled in the art that the antisense ODN combinations of the present invention may also be useful with new chemotherapeutics developed in the future and their effectiveness in combination therewith can be readily tested using methods known in the art and provided herein. The present invention thus contemplates the use of the antisense ODN combinations with both traditional and novel chemotherapeutics.

Examples of TS-inhibiting chemotherapeutics suitable for use with the combinations of the present invention include, but are not limited to 5-FU, 5-FUdR, raltitrexed, methotrexate, capecitabine (an oral form of 5-FU), Alimta® and a topical 5-FU cream (Effudex®). Antisense ODN combinations can also have at least an additive effect with other chemotherapeutics, including, but not limited to hydroxyurea, Tesmilifene®, busulphan, cisplatin, cyclophosphamide, daunorubicin, doxorubicin, melphalan, vincristine, vinblastine, Navelbine®, mitoxantrone, irinotecan (CPT-11) and chlorambucil. Antisense ODN combinations can also have at least an additive effect when used in conjunction with radiotherapy.

Chemotherapeutics are generally administered using a particular therapeutic regimen over a period of weeks or months, and can have deleterious effects on healthy tissues, e.g. by suppressing the bone marrow to some degree or lowering white blood cell counts, resulting in increased risk of infection for patients due to immunosuppression. Other chemotherapeutic agents such as vincristine, which has proved particularly useful as an intravenously administered oncolytic agent in combination with other oncolytic agents for the treatment of various cancers can bring about, following single weekly doses, a wide range of adverse reactions including hair loss, leukopenia, neuritic pain, constipation, and difficulty in walking, abdominal cramps, ataxia, foot drop, weight loss, optic atrophy with blindness, transient cortical blindness, fever, cranial nerve manifestations, paresthesia and numbness of the digits, polyuria, dysuria, oral ulceration, headache, vomiting, diarrhea, and intestinal necrosis and/or perforation. Studies on adverse reactions based on use of Navelbine™ as a single agent indicate granulocytopenia as the major dose-limiting toxicity, although it was generally reversible and not cumulative over time. Mild to moderate peripheral neuropathy manifested by paresthesia and hypesthesia are the most frequently reported neurologic toxicities, occurring in 10% of patients. Mild to moderate nausea occurs in roughly one-third of patients treated with Navelbine™ with a slightly lesser fraction experiencing constipation, vomiting, diarrhea, anorexia, and stomatitis.

While a few compounds exhibiting lessened toxic effects with equal or greater chemotherapeutic activity have been achieved, e.g. 3′,4′-anhydrovinblastine as compared to other vinca alkaloids, the process of synthesising such chemical medicines and screening them for activity can be labour and time intensive. Thus a need remains for relatively non-toxic drugs that can be produced efficiently and provide improved anti-tumour efficacy for the treatment of cancer, or otherwise potentiate the effect of known chemotherapeutics. As indicated above, the present invention provides for a method of reducing the deleterious effects of chemotherapeutic agents by using antisense ODN combinations directed against the TS mRNA to increase the efficacy of the chemotherapeutic(s). Such antisense ODNs are less toxic than many chemotherapeutics (for example, TS antisense ODN 1 is known not to exhibit overt toxicity in animals; Berg et al., 2001, J. Pharmacol. Exp. Ther. 298:477-484). The antisense ODN combinations, therefore, can be used to reduce the amount of chemotherapeutic agent required to elicit an effective anti-cancer response, thereby reducing the incidence of undesirable side effects in patients.

Standard dosage and administration regimens for chemotherapeutic agents are well known in the art and can be found, for example, in the product monographs published in the Compendium of Pharmaceuticals and Specialties, 31^{st} Edition, 1996 (CPS), or the latest edition thereof.

For example, Navelbine® is indicated for the treatment of cancers or tumours such as breast cancer and non-small cell lung cancer. The dosage and administration protocols suggested in the product monograph in the CPS is 30 mg/m² administered weekly via intravenous injection over 6 to 10 minutes. No dose adjustment is required for patients with renal insufficiency, but adjustment to dosage is suggested in accordance with hematologic toxicity or hepatic insufficiency. As another example, Tesmilifene® is used in the treatment of prostate cancer, particularly hormone-unresponsive metastatic prostate cancer, and when administered as an initial intravenous infusion over an approximately one hour period prior to cyclophosphamide treatment, has been shown to potentiate the anti-cancer activity of, and ameliorate the toxicity associated with using, cyclophosphamide or other normally substantially inactive agents. A daily dose of about 240 to about 1200 mg/m² of Tesmilifene® has been shown to afford maximum bone marrow protection and synergy with chemotherapy to kill cancer cells (U.S. Pat. No. 5,863,912).

5-FU has been used as chemotherapeutic for many years both alone and in conjunction with other chemotherapeutics. The following exemplary therapeutic regimens are provided with the understanding that one skilled in the art would appreciate that they may be applied to the situations where 5-FU is used alone or conjunction with another chemotherapeutic. A first exemplary regimen is the Mayo regimen, wherein 5-FU is administered at 425 mg/m ² by intravenous bolus injection daily together with 20 mg/m² leucovorin for 5 days, followed by 3 weeks off. A second exemplary therapeutic regimen consists of administering 200 to 220 mg/mn² 5-FU by continuous infusion over 24 hours once a week. A third means of administering 5-FU consists of using an oral version of the drug, capecitabine (Xeloda®) in two divided doses for a total of 2000-2500 mg/m² daily. A fourth therapeutic regimen consists of shorter, intermittent infusions of 5-FU from between 24 to 120 hours, every week, two weeks, three weeks or four weeks at dosages of 600 mg/m² to 2500 mg/M² per 24 hours.

An exemplary therapeutic regimen for raltitrexed (Tomudex®) is administration at 3 mg/m² once every 3 weeks by bolus injection.

It is contemplated that the antisense ODN combinations directed against TS mRNA provided by the present invention allow for the amount of such chemotherapeutic as described in the above exemplary regimens to be reduced while still preserving a substantially equivalent anti-tumour effect. It is also contemplated that the antisense ODN combinations directed against TS mRNA allow for the enhancement of the efficacy of the above standard protocols. One skilled in the art also appreciates that the chemotherapeutics mentioned above can be used in conjunction with one or more other traditional chemotherapeutic drugs.

Pharmaceutical Preparations

When employed as pharmaceuticals, the antisense oligonucleotides are usually administered in the form of pharmaceutical compositions or formulations. The antisense ODN combinations are generally mixed together in the same composition but may also be administered simultaneously as separate compositions. Such compositions are prepared in a maimer well known in the pharmaceutical art and comprise at least one active compound. The present invention also contemplates pharmaceutical compositions or formulations comprising an expression vector encoding the antisense oligonucleotide of the present invention, which is capable of expressing the antisense ODN in vivo.

In accordance with the present invention, the antisense oligonucleotides may be incorporated into pharmaceutical compositions in the form of pharmaceutically acceptable salts. The term “pharmaceutically acceptable salt” as used herein refers to salts which retain the biological effectiveness and properties of the antisense oligonucleotides of the present invention, and which are not biologically or otherwise undesirable. In many cases, the antisense oligonucleotides of the present invention are capable of forming acid and/or base addition salts by virtue of the presence of amino and/or carboxyl groups or groups similar thereto.

Pharmaceutically acceptable base addition salts can be prepared from inorganic and organic bases. Salts derived from inorganic bases include, but are not limited to, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted allyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstitutedcycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group comprising alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Amines in which two or three substituents, together with the amino nitrogen, form a heterocyclic or heteroaryl group are also suitable.

Examples of suitable amines include, but are not limited to, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts can be prepared from inorganic and organic acids. Salts derived from inorganic acids include hydrochloric acid, hydrobromic acid, sulphuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulphonic acid, ethanesulphonic acid, p-toluene-sulphonic acid, salicylic acid, and the like.

Administration of the Antisense Oligonucleotides

The antisense oligonucleotide combinations of the present invention and pharmaceutical compositions comprising the same may be administered in a number of ways depending upon whether local or systemic treatment of the organism is desired. Administration may be pulmonary, (e.g. by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g. intrathecal or intraventricular, administration. The antisense oligonucleotide combinations of the present invention and pharmaceutical compositions comprising the same may be administered topically in a lotion or cream, for application to the skin in order to treat for example a melanoma.

The antisense oligonucleotide combinations of the present invention may be delivered alone one after the other, or in combination simultaneously, and may be delivered along with a pharmaceutically acceptable vehicle. Ideally, such a vehicle would enhance the stability and/or delivery properties. The present invention also provides for administration of the antisense oligonucleotides or pharmaceutical compositions comprising the antisense oligonucleotides using a suitable vehicle, such as a liposome, microparticle or microcapsule. In various embodiments of the invention, the use of such vehicles may be beneficial in achieving sustained release of the active component, or otherwise protecting the antisense ODNs from nuclease degradation.

For administration to an individual for the treatment cancer, the present invention also contemplates the formulation of the antisense oligonucleotides or pharmaceutical compositions comprising the antisense oligonucleotides into oral dosage forms such as tablets, capsules and the like. For this purpose, the antisense oligonucleotides or pharmaceutical compositions comprising the antisense oligonucleotides can be combined with conventional carriers, such as magnesium carbonate, magnesium stearate, talc, sugar, lactose, pectin, dextrin, starch, gelatin, tragacanth, methylcellulose, sodium carboxymethyl-cellulose, low melting wax, cocoa butter and the like. Diluents, flavoring agents, solubilizers, lubricants, suspending agents, binders, tablet-disintegrating agents and the like can also be employed, if required. The antisense oligonucleotides or pharmaceutical compositions comprising the antisense oligonucleotides can be encapsulated with or without other carriers. In all cases, the proportion of active ingredients in any solid and liquid composition will be at least sufficient to impart the desired activity to the individual being treated upon oral administration. The present invention further contemplates parenteral injection of the antisense oligonucleotides or pharmaceutical compositions comprising the antisense oligonucleotides, in which case they are used in the form of a sterile solution containing other solutes, for example, enough saline or glucose to make the solution isotonic.

For administration by inhalation or insufflation, the antisense oligonucleotides or pharmaceutical compositions comprising the antisense oligonucleotides can be formulated into an aqueous or partially aqueous solution, which can then be utilized in the form of an aerosol.

The present invention also provides for administration of the antisense oligonucleotides in the form of genetic vector constructs that are designed to direct the in vivo synthesis of the antisense oligonucleotides. Within the vector construct, the nucleic acid sequence encoding the antisense oligonucleotide is under the control of a suitable promoter. The vector construct may additionally contain other regulatory control elements. Methods of constructing and administering such genetic vector constructs for in vivo synthesis of antisense oligonucleotides are well-known in the art. In addition, U.S. Pat. No. 6,265,167 teaches an efficient method for the introduction, expression and accumulation of antisense oligonucleotides in the cell nucleus. This method allows the antisense oligonucleotide to hybridize to the sense mRNA in the nucleus, and thereby prevents the antisense oligonucleotide being either processed or transported into the cytoplasm.

The dosage requirements for the antisense oligonucleotides of the present invention or pharmaceutical compositions comprising the antisense oligonucleotides vary with the particular compositions employed, the route of administration, the severity of the symptoms presented and the particular subject being treated. Dosage requirements can be determined by standard clinical techniques, known to a worker skilled in the art. Treatment will generally be initiated with small dosages less than the optimum dose of the compound. Thereafter the dosage is increased until the optimum effect under the circumstances is reached. In general, the antisense oligonucleotides or pharmaceutical compositions comprising the antisense oligonucleotides are administered at a concentration that will generally afford effective results without causing any harmful or deleterious side effects. Administration can be either as a single unit dose or, if desired, the dosage can be divided into convenient subunits that are administered at suitable times throughout the day.

In one embodiment of the invention it is contemplated that the antisense ODN combination directed against the same target mRNA is administered at a total concentration equal to or less than an equivalent effective amount of any single ODN used alone. In another embodiment of the invention, the amount of antisense ODN combination is administered at a total concentration sufficient to chemosensitize cancer cells to a chemotherapeutic agent to an extent at least as much as an equivalent effective amount of the more potent/active ODN of the combination used alone. Typically, when the antisense ODN combination is used as a chemosensitising agent the chemotherapeutic agent is administered within 4 to 24 hours of treatment with the combination. In another embodiment, the antisense ODN combinations are systemically administered to patients, for example, by bolus injection or continuous infusion into a patient's bloodstream.

Therapeutic Uses and Strategies

The antisense ODN combinations directed against TS mRNA of the present invention may be used as part of neo-adjuvant therapy (to primary therapy) or as part of adjuvant therapies where the intention is to cure the cancer in a patient. The present invention contemplates the use of the antisense ODN combinations at various stages in tumour development and progression, including in the treatment of advanced neoplasms (i.e. overt disease in a patient, wherein such overt disease is not amenable to cure by local modalities of treatment, such as surgery or radiotherapy), metastatic disease and locally advanced disease. The use of antisense oligonucleotide combinations directed against TS mRNA to decrease the doses of chemotherapeutic required to treat a particular disease will allow for more prolonged treatments, as may be necessary to treat advanced disease.

Primary therapy is understood to encompass a first line of treatment upon the initial diagnosis of cancer in a patient. Exemplary primary therapies may involve surgery, a wide range of chemotherapies and radiotherapy.

Adjuvant therapy is understood to encompass any therapy, following a primary therapy such as surgery, that is administered to patients at risk of relapsing. Adjuvant systemic therapy is begun soon after primary therapy to delay recurrence, prolong survival or cure a patient. One kind of adjuvant systemic therapy is adjuvant chemotherapy, e.g., using 5-fluorouracil alone or in combination with methotrexate for breast and colorectal cancers, over the course of e.g., four to 24 months. It is contemplated that the antisense ODN combinations can be used in further combination with other chemotherapeutic agents as part of an adjuvant therapy.

In the application of cancer therapies a patient's response status is monitored. Response status refers to measuring what happens to the tumour(s) or lesion(s) under chemotherapy, namely any observed growth (progression of disease), stability, or shrinkage (complete or partial response). Arising out of such monitoring may be the observation of relapse in a patient which may refer to the relapse of a patient with advanced disease. Relapse time is the time from the initial appearance of a primary cancer to the appearance of advanced disease requiring chemotherapy.

The progression of advanced disease is monitored to help evaluate when chemotherapy may be appropriate and may be marked by an increase of at least 25% in the overall sum of measurable lesions as compared to nadir (i. e. best response) and/or the appearance of new lesions following primary therapy. Alternatively, lesions may be found to shrink in size.

Kits

The present invention additionally provides for therapeutic kits containing one or more antisense ODNs that may used in combination, or one or more expression vectors encoding the antisense ODNs, in pharmaceutical compositions for use in the treatment of cancer. The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, for use or sale for human administration.

When the components of the kit are provided in one or more liquid solutions, the liquid solution can be an aqueous solution, for example a sterile aqueous solution. For in vivo use, the antisense ODN may be formulated into a pharmaceutically acceptable syringeable composition. In this case the container means may itself be an inhalant, syringe, pipette, eye dropper, or other such like apparatus, from which the formulation may be applied to an infected area of the subject, such as the lungs, injected into an subject, or even applied to and mixed with the other components of the kit.

### EXAMPLES

Methods

Cell Culture and Chemicals

HeLa cells were obtained from the American Type Culture Collection (Manassas, Va.) and grown in Dulbecco's modified Eagle medium containing 10% fetal bovine serum at 37° C. in a humidified atmosphere of 5% CO₂. All tissue culture reagents, including LipofectAmine® 2000, were from Invitrogen Canada (Burlington, ON, Canada). Raltitrexed (AstraZeneca, Macclesfield, UK) was dissolved in 0.1 M sodium bicarbonate, and 5-FUdR (Sigma, St. Louis, Mo.) was dissolved in water, and diluted in serum-free medium prior to use.

Oligodeoxynucleotides

Fully phosphorothioated ODNs with 2′-methoxy-ethoxy modification on the 6 nucleotides at both the 5′- and 3′-ends were generously provided by Dr. N. Dean (ISIS Pharmaceuticals, Carlsbad, Calif.). ODN 1 (5′-GCCAGTGGCAACATCCTTAA-3′) [SEQ ID NO: 1], ODN 5 (5′-CCAGCCCAACCCCTAAAGAC-3′) [SEQ ID NO:5], and ODN 3 (5′-ACTCAGCTCCCTCAGATTTG-3′) [SEQ ID NO:3], are complementary to nucleotides 1184-1203, 1081-1100 and 1436-1455, respectively, in the 3′ untranslated region of human TS, while ODN 11 (5′-AGCATTTGTGGATCCCTTGA-3′) [SEQ ID NO:11], ODN 6 (5′-GGCATCCCAGATTTTCACTC-3′) [SEQ ID NO:6], and ODN 2 (5′-TTGGATGCGGATTGTACCCT-3′) [SEQ ID NO:2] are complementary to nucleotides 380-399, 419-438 and 1002-1021, respectively, within the protein coding region. The scrambled control ODN 4 (5′-ATGCGCCAACGGTTCCTAAA-3′) [SEQ ID NO:4] has the same base composition as ODN 1, in random order, and is not complementary to any region of human TS. The ODNs were diluted in Milli-Q purified water, and concentrations calculated based on spectrophotometric absorbance readings.

Antisense ODN and Chemotherapeutic Drug Treatments

HeLa cells were plated at 7.5×10⁴ cells per 25-cm² flask in 2 ml of medium. On the following day, the required amounts of ODN and LipofectAmine 2000 were pre-mixed for 15 minutes in serum-free medium. Complete medium was added to yield a 2× transfection mix, and 2 ml of this mixture was added to each flask. Cytotoxic drugs were added to the flasks 4 hours after ODN treatment, in 100 μl volumes. In some experiments, a 1× transfection mix was prepared, and the culture medium on the cells was replaced with 2 ml of the 1× mixture. In this case, cytotoxic drugs were diluted in growth medium to 2× final concentration and added in 2 ml aliquots after 4 hours, and therefore the concentration of ODN was reduced at that time. The ODN concentrations reported indicate the initial ODN concentration, representing the 4-hour pretreatment values. Essentially similar results were obtained with each method. Cells were counted using an electronic particle counter (Beckman Coulter, Hialeah, Fla.) on the day of treatment and 4 days later. Proliferation is expressed as a percentage of that in control flasks using the formula: 100×(experimental final cell number−initial cell number)÷(control final cell number−initial cell number). IC₅₀ values were interpolated from plotted data. Previous studies have shown that, under these conditions, the control scrambled ODN 4 has no effect on TS mRNA or protein levels, or cell proliferation (Ferguson et al., 1999, Br. J. Pharmacol. 127:1777-1786).

ROTA Preparation and Analysis

For isolation of RNA, cells were plated at 1×10⁶ cells per 75-cm² flask in 5 ml of medium. On the following day, ODNs (600 nM) were mixed with 6 μg/ml LipofectAmine 2000 in serum-free medium for 15 minutes at room temperature to yield a 6× transfection mix. One ml of the ODN:lipid mixture was added to each 75-cm² flask to yield final concentrations of 100 nM ODN and 1 μg/ml lipid. RNA was prepared from HeLa cells using the TRIzol reagent (Invitrogen Canada), and quantified using a spectrophometer. For reverse-transcription polymerase chain reaction (RT-PCR), cDNA was prepared from 1 μg of total RNA using Moloney murine leukemia virus reverse transcriptase (Invitrogen Canada) and random hexamer primers. Two percent of the cDNA produced was used as template for PCR with the glyceraldehyde-3-phosphate dehydrogenase (GAPDH) primers GAP-for (5′-TATTGGGCGCCTGGTCACCA-3′) [SEQ ID NO:7] and GAP-rev (5′-CCACCTTCTTGATGTCATCA-3′) [SEQ ID NO:8], or the TS primers TS-for (5′-TTTCGGAGGAGTTGCTGTGG-3′) [SEQ ID NO:9] and TS-rev (5′-TGTGCATCTCCCAAAGTGTG-3′) [SEQ ID NO:10]. PCR cycling parameters were: 3 minutes at 94° C.; followed by 24 cycles of 30 seconds at 94° C., 30 seconds at 58° C., 45 seconds at 72° C.; and a 7 minute 72° C. extension. Products were resolved on 1.5% agarose gels and stained with ethidium bromide. Quantitation of images captured using the ImageMASTER VDS gel documentation system (Amersham Pharmacia Biotech) was done with ImageQuant version 5.1 (Molecular Dymamics).

TS Protein Quantitation

HeLa cells express levels of TS protein that are not readily detectable by western blot techniques using the available antibodies (R. Berg and P. Ferguson, unpublished observations). Therefore, TS was quantitated using a 5-fluorodeoxyuridine monophosphate (5-FdUMP) binding assay (Spears and Gustavsson, 1988, Adv. Exp. Med. Biol. 244:97-106), as previously described (Ferguson et al., 1999, Br. J. Pharmacol. 127:1777-1786). This sensitive assay reliably detects TS at levels as low as 10 fmol/mg total protein (Hoganson et al., 1999, Biochem Pharmacol. 58:1529-1537; Omura et al., 1999, Hepatogastroenterol. 46:985-990).

Flow Cytometry

Cells were treated with 200 nM ODNs and 2 μg/ml lipid, in the same manner as described for RNA preparation, and collected by trypsinization at 48 hours after ODN treatment. Cells were centrifuged for 5 minutes at 500 g, resuspended in phosphate-buffered saline, centrifuged, fixed in 75% ethanol for 15 minutes at room temperature, centrifuged, and washed again. The cells were stained with propidium iodide (0.02 mg/ml in phosphate-buffered saline with 0.1% (vol/vol) Triton X-100 and 0.2 mg/ml deoxyribonuclease-free ribonuclease A) and analysed on a Beckman Coulter XL-MCL flow cytometer. At least 50,000 single cells were analysed for each condition, and the distribution of cells in G₀/G₁, S, and G₂/M cell cycle phases was calculated using MultiCycle software (Version 3.0, Phoenix Flow Systems, San Diego, Calif.).

Statistical Analysis

Statistical differences were determined using Student's t test. A p<0.05 was considered significant. All experiments were performed at least three times.

### DETAILED DESCRIPTION OF THE FIGURES

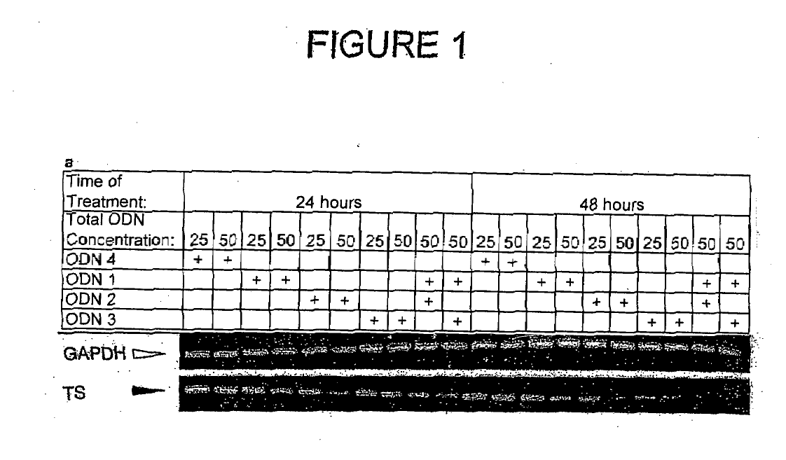

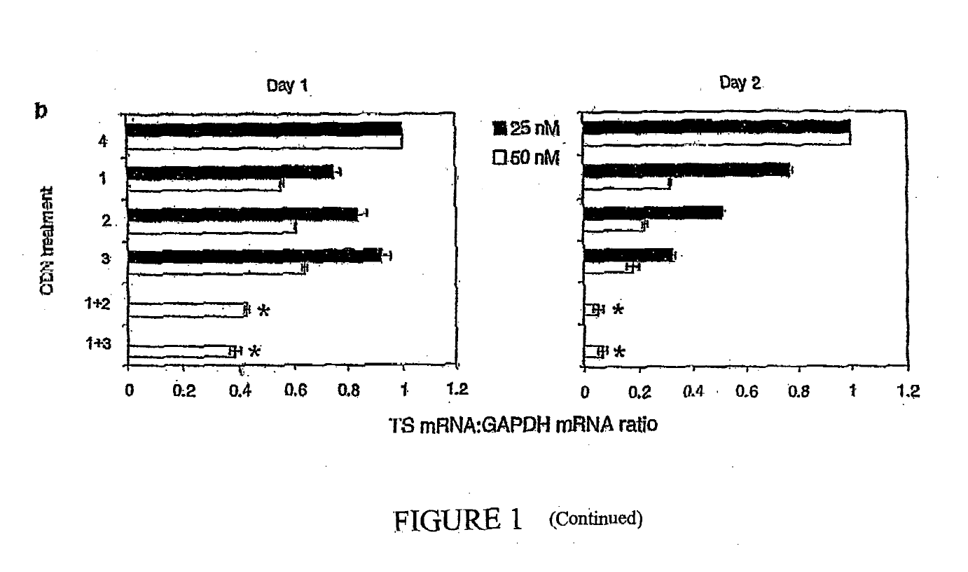

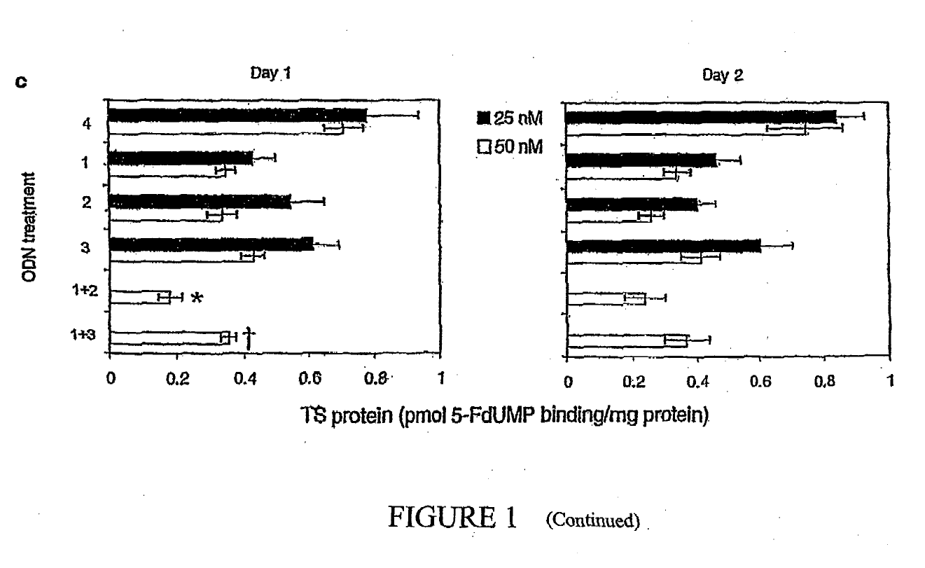

FIG. 1: Quantitation of TS and GAPDH mRNA levels and TS protein levels in HeLa cells treated with ODN combinations. HeLa cells were treated with the indicated ODNs individually (25 or 50 nM) or in equimolar combinations (25 nM each ODN) for 24 or 48 hours. RNA was extracted, and RT-PCR was per-formed and quantitated as described in Methods. (A) Ethidium bromide-stained gel shows GAPDH and TS PCR products from a representative experiment. (B) Quantitation of gel shown in (A) and others showing reductions in TS mRNA following various ODN treatments. (C) HeLa cells were treated for 24 or 48 hours with 25 or 50 nM of the individual ODNs or with ODN combinations (25 nM each ODN), and TS protein levels were quantitated by 5-FdUMP binding as described in Methods. Asterisks tindicate significant differences (p<0.05) compared to cells treated with individual ODNs (50 nM). A dagger indicates a significant difference only compared to cells treated with ODN 3 alone.

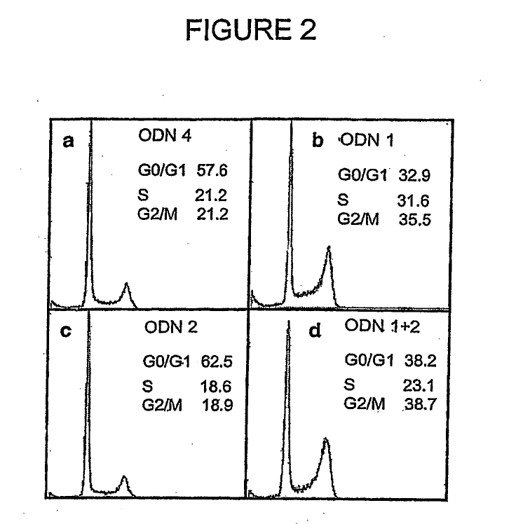

FIG. 2: Flow cytometric analysis of cell cycle profiles in HeLa cells treated with ODN combinations. HeLa cells were treated with 200 nM of (A) ODN 4, (B) ODN 1 or (C) ODN 2; or (D) with 100 nM each of ODNs 1+2 for 48 hours, and collected for analysis by flow cytometry as described in Methods. Insets show the fraction of cells in G₀/G₁, S, and G₂/M, determined using MultiCycle software.

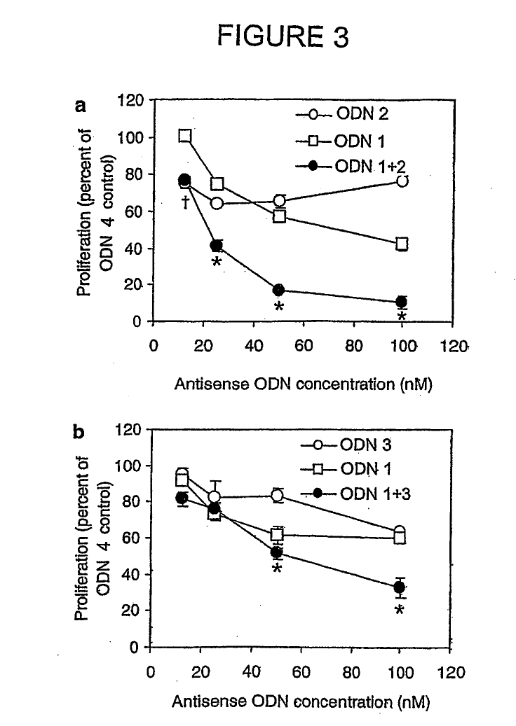

FIG. 3: Combinations of ODNs inhibit proliferation of HeLa cells more effectively than individual ODNs. HeLa cells were treated with TS antisense ODNs 1, 2, and 3 individually or in a 1:1 ratio to yield the combined concentrations indicated, and counted 4 days later. Total ODN concentration in each treatment was adjusted to 100 nM using the control scrambled ODN 4. Shown are the mean proliferation (±SD) relative to cells treated with 100 nM ODN 4, for 3 flasks from representative experiments. Asterisks indicate significant decreases in proliferation (p<0.05, Student t test) when compared to cells treated with either antisense ODN alone. A dagger indicates a significant decrease in proliferation only when compared to cells treated with ODN 1 alone.

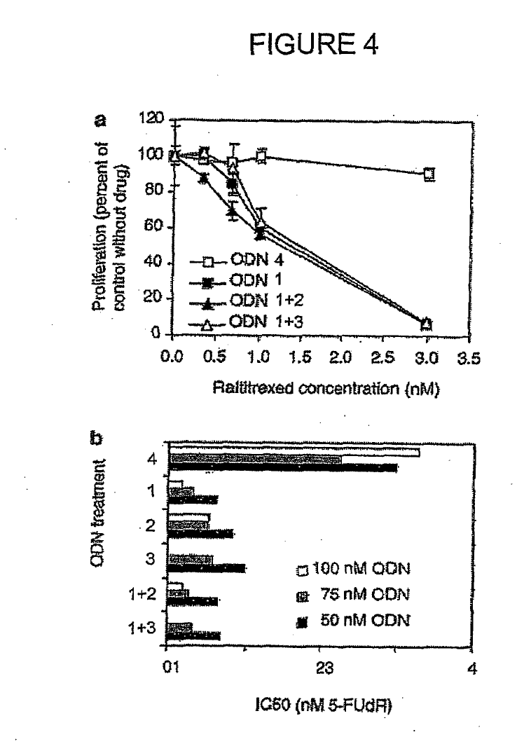

FIG. 4: Sensitivity of HeLa cells to raltitrexed and 5-FUdR following treatment with combinations of TS antisense ODNs. (A) HeLa cells were treated with 100 nM (total) of the indicated ODNs for 4 hours, then the indicated concentration of raltitrexed was added, and the cells were counted 4 days later. Shown are the mean proliferation (±SD) relative to cells treated with each ODN mixture in the absence of raltitrexed for 3 flasks from a representative experiment. (B) HeLa cells were treated with the indicated total concentration of ODN for 4 hours, then with varying concentrations of 5-FUdR. Shown are the average of 5-FUdR IC₅₀ values from at least 3 experiments.

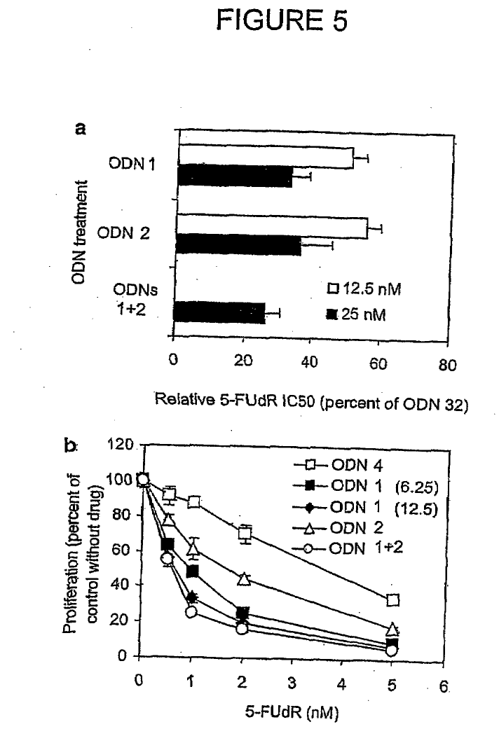

FIG. 5: Lower concentrations of ODN combinations cannot sensitize HeLa cells to 5-FUdR, compared to ODN 1 alone. (A) HeLa cells were treated for 4 hours with ODN 1 or ODN 2 at 12.5 or 25 nM, or in combination (12.5 nM each ODN), followed by 0 to 10 nM 5-FUdR. The IC₅₀ of 5-FUdR for cells treated with antisense ODNs, relative to cells treated with ODN 4, is shown (average of 3 experiments ±SD). (B) Cells were treated with 6.25 nM of ODN 1, 12.5 nM of ODN 1 or ODN 2, or 6.25 nM each of ODNs 1+2 for 4 hours. Total ODN concentration was equalized at 50 nM using control ODN 4. The indicated concentration of 5-FUdR was added, and cells counted 4 days later. Average relative proliferation (±SD) from 3 flasks from a representative experiment is shown.

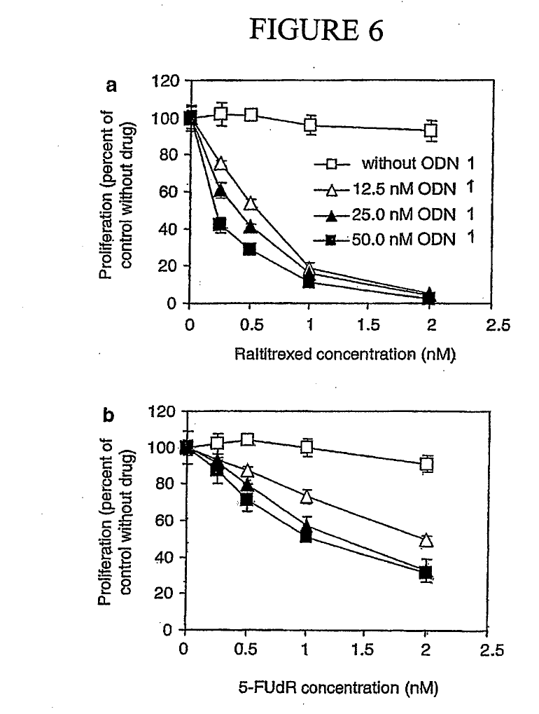

FIG. 6: Enhanced sensitivity of HeLa cells to lower doses of raltitrexed and 5-FUdR following treatment with various concentrations of ODN 1. HeLa cells were treated with the indicated concentration of ODN 1, mixed with ODN 4 to total 50 nM, for 4 hours. The indicated concentration of (A) raltitrexed or (B) 5-FUdR was added, and the cells were counted 4 days later. Shown is the mean proliferation (±SD) relative to cells treated with each concentration of ODN 1 in the absence of drug for 3 flasks from a representative experiment.

FIG. 7: Predicted single-stranded regions in TS mRNA. Predicted foldings of TS mRNA were obtained using mfold version 3.1 (Mathews et al., 1999, J. Mol. Biol. 288:911-940; Zuker et al, In: Barciszewski J, Clark B F C, eds. RNA Biochemistry and Biotechnology. New York, N.Y.: Kluwer Academic Publishers; 1999, 11-43). The plot shows the number of foldings (out of 29 unique foldings obtained) in which each base is single-stranded, averaged over a 51-base window. In the upper diagram, thin horizontal lines represent the 5′- and 3′-untranslated regions, while the thick portion is the coding region flanked by the translation start site and stop codon (boxes). Vertical arrows indicate the location of sequences complementary to TS antisense ODNs, while vertical bars indicate the exon boundaries in the TS gene.

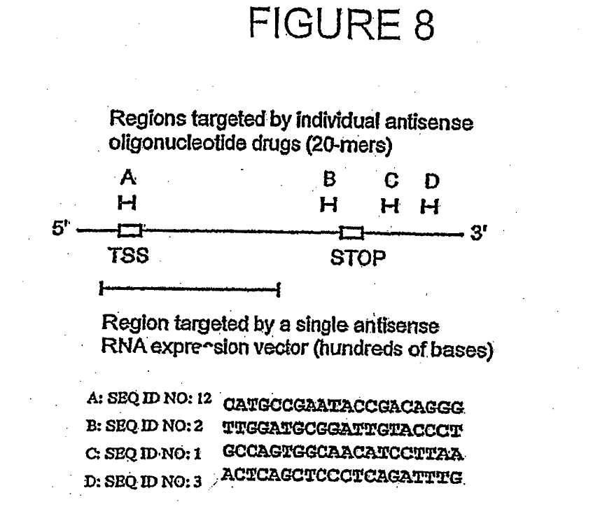

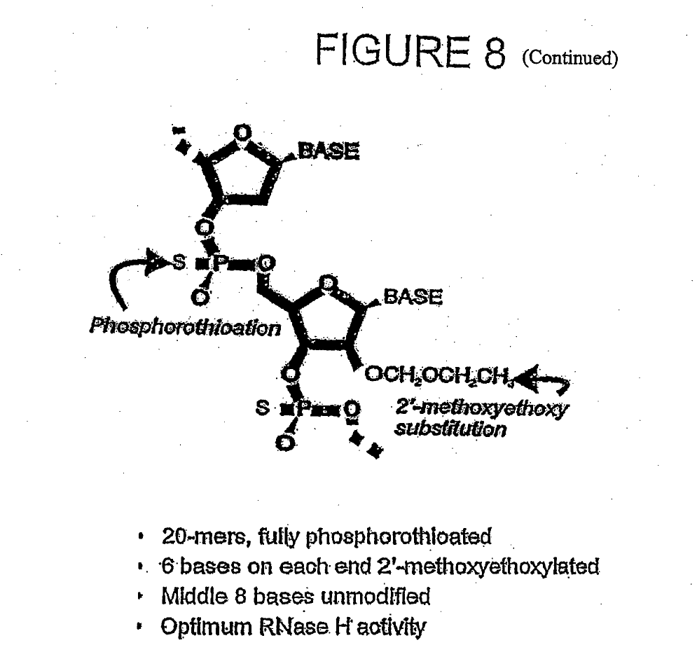

FIG. 8: Schematic representation of ODN structures. Schematic diagram showing the location of sequences targeted by antisense ODNs. Treatment with ODN 11 (A), or an antisense RNA expression vector targeting the translation start site (TSS) increases TS gene transcription. Structural modifications to ODN 2 (B), ODN 1 (C), and ODN 3 (D) are shown. These ODNs downregulate TS mRNA and protein levels, but have differential effects on cell cycle progression and cell proliferation.

### Example 1

### ODN Combinations Enhance Antisense Downregulation of TS

To test the hypothesis that combinations of antisense ODNs might be more effective than single ODNs, several pairs of antisense ODNs targeting human TS mRNA were analyzed for their effects on HeLa cells in vitro. TS antisense ODN 1, used alone, effectively inhibits HeLa cell proliferation compared to the scrambled control ODN 4 (Table 3) (Ferguson et al., 1999, Br. J. Pharmacol. 127:1777-1786). In initial experiments, ODN 1 (50 nM) was combined with equimolar amounts of one of a panel of antisense ODNs targeting different regions of TS mRNA, and the results compared to those obtained when ODN I was combined with control ODN 4 (an ODN that does not target TS mRNA or any other known human sequences). Certain partner ODNs enhanced the antiproliferative response while others did not (Table 3); subsequent experiments focussed on treatment with ODN 1 in combination with ODN 2 or 3.

HeLa cells were treated with these pairs of ODNs, and RT-PCR analysis performed to measure reductions in TS mRNA level. Treatment with combinations of TS antisense ODNs 1+2 and ODNs 1+3 resulted in enhanced reductions of TS mRNA levels, compared to treatment with each ODN used as a single agent (FIG. 1A and 1B). Decreased TS message levels were reflected in reduced TS protein and activity levels, measured by [6³H]5-FdUMP binding, as early as 24 hours after treatment with ODNs 1+2 (FIG. 1C). By 48 hours post-treatment the TS level in cells treated with the ODN combinations was equivalent to those treated with the individual ODNs but still significantly lower than in cells treated with the control scrambled ODN 4. Thus, TS antisense ODN combinations downregulate both TS mRNA and protein, and do so more effectively than individual ODNs at an early time following treatment.

TABLE 3
Enhancement of the antiproliferative activity of ODN 1 by combination
treatment with additional TS antisense ODNs.

| | | Inhibition of Cell | Inhibition in |
|---|---|---|---|
| | | Proliferation^{c} | Combination with |
| ODN | Cell Number | (relative to ODN 4- | ODN 1 (% |
| Combination^{a} | (SEM)^{b} | treated) | increase^{d}) |
| 4 alone | 9.65 (0.34) | | |
| 1 + 4 | 4.94 (0.91) | 55 | |
| 1 + 11 | 2.82 (0.51) | 80 | 45 |
| 1 + 6 | 3.77 (0.58) | 69 | 25 |
| 1 + 2 | 1.67 (0.28) | 94 | 69 |
| 1 + 5 | 5.06 (0.55) | 54 | −3 |
| 1 + 3 | 4.26 (0.89) | 63 | 14 |

^{a}ODN 4 alone was used at 100 nM; otherwise mixtures were 50 nM of each ODN.
^{b}Number of cells (×10⁻⁵) per 25-cm² flask after 4 days of ODN treatment (average of 3 flasks in a representative experiment). Starting cell number (day 0) was 91300.
^{c}Percent inhibition, calculated using the formula: 100 − 100 × (experimental final cell number − initial cell number) ÷ (control final cell number − initial cell number)
^{d}Calculated as a percentage of the inhibition achieved with ODN 1 + 4, using the formula: 100 × (combination antisense ODN − ODN 1 + 4) ÷ (ODN 1 + 4).

### Example 2

### Antisense ODN Combinations Exhibit Enhanced Antiproliferative Activity

Inhibition of HeLa cell proliferation by ODN 1 treatment has previously been shown to be accompanied by transient G₂/M cell cycle arrest (Berg et al., 2001, J. Pharmacol. Exp. Ther. 298:477-484). To assess the ability of ODN combinations to induce cell cycle arrest, a flow cytometric analysis was used. FIG. 2 shows that a 48-hour treatment with ODN 1 alone or with the combination of ODNs 1+2 induced G₂/M arrest to a similar extent, but that ODN 2 alone had no significant effect on the cell cycle profile. ODN 2 is representative of several other TS antisense ODNs that, when used as single agent treatments, had little or no dose-dependent effects on cell cycle or cell proliferation but effectively enhanced the cytotoxicity of raltitrexed and 5-FUdR (P. Ferguson and R. Berg, unpublished observations).

The ODN combinations were then examined for the ability to inhibit HeLa cell proliferation over a range of ODN concentrations. The combinations of ODNs 1+2 and ODNs 1+3 enhanced inhibition of HeLa cell proliferation, compared to the effect of exposure to each of the ODNs used individually (FIG. 3A and 3B). The combination of ODNs 2+3 was equivalent to ODN 3 alone, and the triple mixture was no more effective than either double mixture (data not shown). Furthermore, varying the ratio of ODN 1:partner ODN from 1:7 to 7:1 did not further augment the enhanced activity (data not shown).

### Example 3

### Antisense ODN Combinations Chemosensitize HeLa Cells to Anti-TS Drugs

The capacity of combinations of TS antisense ODNs to inhibit proliferation more effectively than individual ODNs raised the possibility that ODN combinations would similarly be more effective in enhancing tumour cell sensitivity to TS-targeting drugs. Therefore, the ability of these combinations of ODNs to enhance the sensitivity of HeLa cells to the TS inhibitors raltitrexed and 5-FUdR was examined. Compared to treatment with the control scrambled ODN 4, treatment with the combinations of ODNs 1+2 and ODNs 1+3 increased the cytotoxicity of raltitrexed (FIG. 4A) and 5-FUdR (FIG. 4B). However, the degree of enhancement was not different than that achieved by treatment with ODN 1 alone.

It was hypothesized that the observed lack of differential chemosensitising effects between individual and combination ODNs might be related to the ODN concentration at which testing was done. That is, if cells were treated with lower ODN concentrations, differences might become apparent. The combination ODN strategy was therefore assessed for efficacy in sensitizing HeLa cells to 5-FUdR cytotoxicity using lower concentrations of ODNs. Sensitization to 5-FUdR, achieved using an equimolar combination of ODNs 1+2 (12.5 nM each), was not significantly different from that achieved with either of the individual ODNs used alone at 25 nM (FIG. 5A). Similarly, the drug sensitivity of cells treated with the combination of ODNs 1+2 (6.25 nM each) was equivalent to cells treated with ODN 1 alone (12.5 nM) (FIG. 5B), At still lower ODN concentrations (i.e., 1 and 2 nM), treatment with individual or combinations of TS antisense ODNs did not enhance sensitivity to 5-FUdR; the dose-response curves were indistinguishable from control ODN 4-treated cells (data not shown).

In further control experiments to examine the dose-response following individual ODN treatment, HeLa cells were treated with 12.5 to 50 nM ODN 1, followed by various doses of raltitrexed or 5-FUdR. Treatment with ODN 1 at each of these concentrations effectively sensitized HeLa cells to the cytotoxicity of raltitrexed, and while the cytotoxicity of 5-FUdR was increased at 12.5 nM, maximal chemosensitivity was achieved with 25 nM ODN 1 (FIG. 6). In contrast, only the 50 nM dose of ODN 1 significantly inhibited cell proliferation in the absence of chemotherapeutic drug in these experiments, by approximately 20% (data not shown).

### SUMMARY

Targeting different regions of TS mRNA with antisense ODNs has diverse physiological consequences, not always in accord with antisense-mediated reduction in TS mRNA. TS antisense ODN 1 (targeting the 3′-untranslated region) downregulates TS mRNA and protein, inhibits cell proliferation and sensitizes cells to drugs that inhibit TS enzyme activity (Berg et al., 2001, J. Pharmacol. Exp. Ther. 298:477-484; Ferguson et al., 1999, Br., Pharmacol. 127:1777-1786). In contrast, antisense targeting the translation start site increases TS gene transcription (DeMoor et al., 1998, Exp Cell Res. 243:11-21). Furthermore, certain TS antisense ODNs that downregulate TS mRNA to a similar extent as ODN 1 increased drug sensitivity without affecting cell proliferation on their own (P. Ferguson and R. Berg, unpublished observations). Differences in the biological consequences following antisense targeting of different regions of the same mRNA led to the prediction that combinations of TS antisense ODNs, each targeting distinct regions of TS mRNA, would inhibit or enhance one or more of their downstream effects. As indicated herein, treatment of HeLa cells with some combinations of antisense ODNs targeting human TS mRNA resulted in enhanced reductions in TS mRNA levels and enhanced inhibition of proliferation, compared with the ODNs used individually. An embodiment of the invention therefore relates to improved antisense effects through use of multiple antisense ODNs against the same target mRNA.

TS expression is regulated at multiple transcriptional and post-transcriptional levels. TS antisense ODNs 1, 2, and 3, used individually, reduced TS mRNA and protein levels to a similar extent. In response to 24 hour treatment with combined ODNs 1+2, reduction in TS protein levels was greater than that due to treatment with either of the ODNs alone: an effect that was matched by decreased TS mRNA levels (FIG. 1). Therefore, there was a more-than-additive antisense effect of this combination at both the mRNA and protein level. However, 48 hours post-treatment, there was no enhanced downregulation of TS protein in spite of continued decrease in TS mRNA. The combination of ODNs 1+3 enhanced reduction of TS mRNA, similar to the combination of ODNs 1+2 but, unlike 1+2, the 1+3 combination did not enhance TIS protein downregulation: the effect was only additive. This suggests that different combinations of antisense ODNs affected TS expression at different control points. Presumably mRNA stability and translation, and possibly protein stability, were affected by the 1+2 combination. On the other hand, the 1+3 combination enhanced only events regulating mRNA levels (for example, mRNA degradation) without enhancing effects on TS mRNA translation or TS protein stability. These differential effects of simultaneously targeting different TS mRNA regions suggests a role for TS mRNA in regulating expression at those different levels. The transcription start site, through its interaction with TS protein, has been suggested to have an autoinhibitory role (Chu et al., 1991, Proc Natl Acad Sci USA 88:8977-898; Chu et al. 1994, Mol Cell Biol. 14:207-213). The potential for other TS mRNA regions to regulate TS expression has only been revealed by antisense targeting, and is currently being explored. Individual or combination ODN treatments, in all cases, enhanced sensitivity to TS-targeting drugs to the same degree, in accord with reduction in TS protein levels, regardless of the enhanced reduction in TS mRNA. This suggests, not unexpectedly, that TS protein (but not TS mRNA) is the essential factor mediating drug sensitivity.

The correlation between enhanced reduction in TS mRNA levels mediated by combined ODNs, and enhanced antiproliferative effects, suggested that TS mRNA may mediate a function relevant to proliferation, irrespective of TS protein. However, while treatment of HeLa cells with individual ODN 1 or ODN 2 reduced TS mRNA and protein levels, and sensitized cells to 5-FU and raltitrexed to similar extents, ODN 2 treatment had no effect on cell cycle or proliferation, whereas ODN 1 induced G₂/M arrest and inhibited cell proliferation. In fact, the TS mRNA region complementary to ODN 1 is the only one we have been able to target to induce G₂/M arrest, although targeting several others inhibited cell proliferation (data not shown). Interestingly, ODN 1 targets a region of exceptionally low potential for secondary structure in TS mRNA (FIG. 7) and this may be of significance with respect to potential physiological interactions of TS mRNA with other molecules.

G₂/M arrest may not be essential for ODN 1 to exert its antiproliferative effect. Combined treatment with ODNs 1+2 induced G₂/M arrest similar to ODN 1 alone (FIG. 2), but was more potent at inhibiting proliferation (FIG. 3). Therefore, these two events were not necessarily associated. However, the method used to assess proliferation did not distinguish between effects on cell cycle and cell death. Although ODN 1 treatment has been shown not to induce apoptosis in HeLa cells (Berg et al., 2001, J. Pharmacol. Exp. Ther. 298:477-484), treatment with ODNs 2, 3 or combinations of ODNs might induce cell death and contribute to reduced cell numbers.

Moreover, RNase H-mediated cleavage of alternate mRNA targets and aptameric effects of TS antisense ODNs on TS or other unidentified proteins are formally possible. Global analysis of gene expression changes following ODN administration using cDNA and oligonucleotide microarrays is one strategy to assess the former possibility. To address potential aptameric effects, proteins that interact with these ODNs are being identified in cultured cells and cell lysates. Finally, to dissect cytostatic and cytotoxic effects, clonogenic survival of cells treated with ODNs (alone and in combinations), with and without raltitrexed and 5-FUdR, is being measured. This is an important measure of potential clinical utility of combination therapy since the preferred effect of therapy is to eliminate malignant cells rather than reduce proliferation.

Combining ODNs to enhance antitumour effects may be effective in minimizing the potential impediment to successful therapy presented by poor penetration of antisense ODNs into a solid tumour mass. Furthermore, the observation that low ODN concentrations (<10 nM) sensitize HeLa cells to drugs in vitro suggests that even moderate entry of ODNs into solid tumours may be sufficient to chemosensitize them. Therapies incorporating antisense ODNs to downregulate TS could enhance the effects of lower doses of cytotoxic drugs (e.g., raltitrexed or 5-FUdR) and reduce the danger of systemic toxicity.

The instant application describes the effects of combined ODNs targeting the same mRNA on tumour cell proliferation and drug sensitivity. The rationale in targeting TS with antisense ODNs is that reduction in TS mRNA and protein has two effects: reduced proliferation as a consequence of decreased thymidylate production, analogous to the effect of treatment with conventional TS-targeting chemotherapeutic drugs; and sensitization of tumour cells (including drug-resistant and TS-overexpressing cells) to existing and novel TS-targeting drugs. Combinations of TS antisense ODNs used in conjunction with TS-targeting drugs (for example, ODNs 1+2 with raltitrexed) would be expected to have a greater overall antitumour effect than individual antisense ODNs. Decreases in tumour cell numbers would be achieved by both antiproliferative effects and enhancement of TS-targeting drug action. The results presented here suggest that use of multiple antisense ODNs, with or without traditional drugs in combination treatment, may improve the efficacy of antisense therapy.

### Example 4

### Combinations of Antisense Agents

Background:

Gene silencing by antisense is impeded by poor delivery of antisense molecules to and into target cells. In vivo delivery of antisense reagents in a stable form and appropriate dose to the appropriate target site can be a considerable challenge (Lebedeva I, Benimetskaya L, Stein C A, Vilenchik M A. Cellular delivery of antisense oligonucleotides. Eur. J. Pharm. Biopharm. 2000;50:101-119; Shi F, Hoekstra D. Effective intracellular delivery of oligonucleotides in order to make sense of antisense. J. Control. Release. 2004;97:189-209). A need remains to develop antisense agents and delivery strategies that provide for enhanced tissue penetration and cellular uptake.

Intracellular delivery of antisense agents and effectiveness in downregulating targets is variable among agents, e.g. among molecules of different molecular weight and among molecules with different backbone chemistries, however, the relationship is not direct (i.e., intracellular delivery and accumulation does not change in strict accord with increasing or decreasing molecular weight). Rather, antisense activity is not determined solely by molecular weight or backbone chemistry, but an interplay of both factors (Sundaram S, Lee L K, Roth C M, Interplay of polyethyleneimine molecular weight and oligonucleotide backbone chemistry in the dynamics of antisense activity. Nucleic Acids Res. 2007;35(13):4396-408).

We have observed that select combinations of 20-mer antisense oligodeoxyribonucleotides (AS-dODNs) (2-MOE and 2-MO chemistries) targeting thymidylate synthase (TS) mRNA at non-contiguous regions both with the coding region and in the 3 ′-untranslated region are more effective at reducing thymidylate synthase (TS) mRNA abundance and inhibiting cell proliferation than the individual AS-dODNs used alone. In contrast to the effects on cell proliferation, the enhanced sensitivity to anti-TS chemotherapeutic drugs (i.e., raltitrexed and 5-fluorodeoxyuridine) that is achieved by treatment with individual AS-dODNs was not further augmented by combined AS-dODN treatment. This suggests that the AS-dODNs combinations tested inhibit an alternative function of TS mRNA or protein, distinct from thymidylate production. The results are evidence that the novel use of multiple AS-dODNs that target different regions of the same mRNA represents a general strategy to improve anti sense effectiveness (Berg R W, Ferguson P J, Vincent M D, Koropatnick D J. A “combination oligonucleotide” antisense strategy to downregulate thymidylate synthase and decrease tumor cell growth and drug resistance (Cancer Gene Ther. April 2003;10(4):278-86), herein incorporated by reference).

Targeting thymidylate synthase mRNA with two or more antisense agents, each composed of fewer than 20 bases, will lead to enhanced uptake of those antisense agents with concomitant increased intracellular antisense molecule levels. While antisense agents of less than 20 bases bind target mRNA with decreased specificity, and that decrease in specificity is correlated with the degree of shortening, specificity may be maintained by exposing target cells (e.g. neoplastic cells) and tissues (e.g. a tumor mass) to a combination treatment with two or more antisense agents of diminished length and targeting non-contiguous mRNA sequences. The antisense treatment will be effective when two or more antisense molecules are simultaneously associated with target sequences (because of synergistic or additive effects resulting from multiple antisense agents associating with the same target mRNA), while “off-target” associations of smaller numbers of the antisense agents with non-targeted mRNAs will be ineffective at inducing degradation or inhibiting translation of those non-target mRNAs (because such off-target associations will involve fewer antisense agents associating with the same non-targeted mRNA). The efficacy of this antisense agent strategy will be valid both for treatment of isolated cells in culture, for primary cells and/or tissue masses in whole organisms treated systemically or in a localized manner with combinations of antisense agents.

In one embodiment of the invention, a combination of two or more antisense agents is provided, complementary to one or more target mRNAs, wherein at least one agent consists of less than 20 nucleotides or nucleotide bases. In another embodiment of the invention, the combination comprises at least one antisense agent with fewer than 14 nucleotides or nucleotide bases. In yet another embodiment of the invention, the combination comprises at least one antisense agent consists of 10 nucleotides or nucleotide bases. In yet a further embodiment of the invention, the combination comprises two or more antisense agents, each with no fewer than 7 nucleotides or nucleotide bases.

Exemplary combinations of antisense agents targeting thymidylate synthase mRNA or another target mRNA may comprise one or more agents that do not alone have an antiproliferative effect or otherwise sensitize a cell to a chemotherapeutic, so long as there is at least one agent of the combination that does. Exemplary agents that target thymidylate synthase mRNA and exhibit an antiproliferative effect or otherwise sensitize a cell to a chemotherapeutic are antisense agents according to any one of SEQ ID Nos. 1, 2, 3, 13, 14, 15, 16, 17, 18 or 19.

In one embodiment of the invention, a combination of antisense agent is provided that comprises at least one antisense agent complementary to a thymidylate synthase mRNA, that consists of fewer than 20 nucleotides or nucleotides bases. In another embodiment of the invention, the combination of antisense agents comprises at least one antisense agent complementary to a thymidylate synthase mRNA that comprises 20 nucleotides or nucleotide bases. In yet another embodiment of the invention, the combination of antisense agents comprises at least one antisense agent complementary to a thymidylate synthase mRNA and at least one antisense agent complementary to a target mRNA other than a thymidylate synthase mRNA.

Selecting sequences for antisense agents for use in the combinations of the invention, wherein at least one agent of the combination consists of less than 20 nucleotides or nucleotide bases is done by in silico screening. This will ensure that the antisense agents would be sufficiently non-complementary to non-target RNAs so that they would remain specific for a thymidylate synthase mRNA or other mRNA that is the intended target of one or more of the antisense agent(s) for use in combination. In addition, analysis is done of the relative content of specific bases to maximize affinity of the antisense molecules for their target sequences (i.e., different bases have different affinities for their complementary bases, with GC pairing stronger than AU).

The antisense agents for use in the combinations of the invention may have variable chemistries—including but not limited to, phosphorothioates (PS), 2′-O-methyl RNA (OMe), 2′-O-methoxy ethyl RNA (MOE), peptide nucleic acids (PNA), N3′-P5′-phosphoroamidates (NP), 2′-fluoro-arabino nucleic acids (FANA), locked nucleic acids (LNA), morpholino phosphoroamidate (MP), cyclohexene nucleic acid (CeNA), and tricyclo-DNA (tcDNA)—where all combined antisense agents are of the same or different chemistries. Cells may get saturated taking up a particular type of molecule and such saturation can be circumvented by using antisense molecules with different chemistries (e.g. backbones). This is an alternative combination antisense agent design strategy that can be used on its own or together with the strategy of using in the combinations of the invention at least one antisense agent consisting of less than 20 nucleotides or nucleotide bases in length.

In one embodiment of the invention, a combination is provided comprising at least two antisense agents of different chemistries. In another embodiment of the invention, the combination of the at least two antisense agents of different chemistries, includes at least one antisense agent consisting of less than 20 nucleotides or nucleotide bases in length. In yet another embodiment of the invention, the at least two antisense agents of different chemistries are complementary to contiguous regions of a target mRNA. In yet a further embodiment of the invention, the at least two antisense agents of different chemistries are complementary to non-contiguous regions of a target mRNA.

Antisense “Gapmer” Agents

Antisense oligonucleotide “gapmers” have been reported to have increased specificity 30 for target mRNAs when the “gap” within antisense oligonucleotides is of reduced length and involves a minimal number of bases (Takagi-Sato M, Tokuhiro S, Kawaida R, Koizumi M. Fine-tuning of ENA gapmers as antisense oligonucleotides for sequence-specific inhibition. Oligonucleotides. 2007 Fall;17(3):291-301). The use of antisense oligonucleotide gapmers of diminished overall length and diminished numbers of bases involved in gap sequences may have enhanced capacity to enter cells in vitro and in vivo, and have increased antisense effectiveness. The gapmers have catalytic capacity, that is, one antisense molecule can bind to multiple RNA molecules one after the other, thus amplifying effect. In addition, the gap improves specificity of cleavage by RNAse H, an advantage when seeking to enhance specificity for short antisense molecules that have reduced specificity compared to longer antisense molecules. When gap sequences are at one end of the antisense oligonucleotide molecule rather than the center (hemimers) or when multiple gaps are inserted into antisense oligonucleotides (gap-disabled antisense oligonucleotides), reducing the length of the gap to enhance specificity will enhance overall effectiveness of combining multiple antisense molecules targeting different non-contiguous mRNA sequences.

In one embodiment of the invention, a combination of antisense agents is provided comprising at least one antisense gapmer agent. In another embodiment of the invention, the antisense gapmer agent is less than 20 nucleotides or nucleotide bases in length, In yet another embodiment of the invention, the antisense gapmer agent comprises a gap of at least four bases.

Antisense RNA Agents

Antisense RNA (shRNAs, RNAi, and siRNA) has reduced capacity to enter cells, largely because of the high molecular weight resulting from double-strandedness. Reducing the length of antisense RNAs of multiple types will also enhance the penetration of such agents into tissues and uptake into cells. In one embodiment of the invention, a combination of antisense agents is provided comprising at least one antisense RNA directed to a target mRNA. In another embodiment of the invention, the at least one antisense RNA consists of less than 20 nucleotides or nucleotide bases in length. In yet another embodiment of the invention, the at least one antisense RNA is directed to a thymidylate synthase mRNA.

The following siRNAs were obtained from Dharmacon RNA Technologies (Lafayette, Colo.). All siRNAs were obtained in annealed and desalted form, then dissolved in siRNA buffer (supplied by Dharmacon) to obtain a 10 μM working solution.

a) siRNA duplex targeting TS (targeting bases 868-886 of TS mRNA based on GenBank Accession No. NM_{—}001071): antisense (5′-GAUUCAGGUAAAUAUGUGCWU, SEQ ID NO:20) sense (5′-GCACAUAUUUACCUGAAUCUU, SEQ ID NO:21);

b) Control (nontargeting) siRNA duplex (SC): designed by Dharmacon to have ≧4 mismatches with all known human mRNAs.

Delivery of Antisense Agent Combinations

The effectiveness of the antisense agent combinations for improved tissue penetration and/or cellular delivery is assessed in vitro after administration with or without agents to enhance general uptake into cells and/or target them to specific cells (including but not limited to cationic liposomes, microbubbles, and liposomes and immunoliposomes) by:

a) enhanced capacity to reduce TS mRNA and/or other RNAs against which the antisense molecules are targeted (by quantitative real-time PCR),

b) enhanced capacity to reduce TS protein and/or other RNAs against which antisense molecules are targeted (by enzyme-linked immunoassay, immunoblot, and other appropriate methodology to measure levels of specific proteins encoded by mRNAs), and

c) enhanced capacity to sensitize cells to chemotherapeutic drugs (by measurement of induction of apoptosis and/or cytostasis).

Enhanced in vivo effectiveness is assessed by:

a) enhanced capacity to reduce target mRNA and/or other RNAs against which the antisense molecules are targeted in human tumour xenografts and in spontaneously-occurring, induced, and xenografted non-human tumours, after delivery to whole organisms (including but not limited to immunocompromised and immune-competent rodents). The antisense molecules are delivered in vivo with or without association with various vehicles for delivery of molecules to target organs and cells (including but not limited to liposomal and immunoliposomal preparations, microbubbles, and direct attachment to targeting antibodies), and

b) enhanced capacity to inhibit growth of human and non-human tumours in appropriate animal models, when antisense molecule combinations are delivered alone or in combination with non-antisense drugs. Increased capacity to inhibit growth and/or induce tumour regression, alone or in combination with non-antisense drugs, will be tested in both nascent tumours (i.e., immediately after injection of tumour cells, or prior to appearance of spontaneous or induced tumours, in appropriate animal models) and established tumors (i. e., those allowed to grow to detectable and measurable size prior to treatment).

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A pharmaceutical composition comprising two or more antisense oligonucleotides complementary to a thymidylate synthase mRNA or analogues of said oligonucleotides and a pharmaceutically acceptable carrier.

2. The pharmaceutical composition according to claim 1, wherein at least one of said antisense oligonucleotides or analogues thereof comprises at least 5 consecutive nucleotides from the nucleic acid sequences as set in forth in any one of SEQ ID NOs: 1, 2, 3, 5, 6, 11 and 13-19.

3. The pharmaceutical composition according to claim 1, wherein said antisense oligonucleotides or analogues thereof comprise at least 5 consecutive nucleotides from the nucleic acid sequences as set in forth in any one of SEQ ID NOs: 1, 2, 3, 5, 6, 11 and 13-19.

4. The pharmaceutical composition according to claim 1, wherein said analogues comprise one or more phosphorothioate linkages.

5. The pharmaceutical composition according to claim 1, wherein said analogues comprise one or more 2′-methoxy-ethoxy substituted nucleotide.

6. The pharmaceutical composition according to claim 1, further comprising one or more chemotherapeutic agents.

7. A method of using a composition comprising two or more antisense oligonucleotides complementary to a thymidylate synthase mRNA or analogues of said oligonucleotides in the treatment of cancer, comprising the step of administering said composition to a mammal.

8. The method according to claim 7, wherein the composition is administered in conjunction with one or more chemotherapeutic agents.

9. The method according to claim 8, wherein the composition sensitizes neoplastic cells to at least one of said one or more chemotherapeutic agents.

10. The method according to claim 7, wherein at least one of said antisense oligonucleotides or analogues thereof comprises at least 5 consecutive nucleotides from the nucleic acid sequences as set in forth in any one of SEQ ID NOs: 1, 2, 3, 5, 6, 11 and 13-19.

11. The method according to claim 7, wherein said antisense oligonucleotides or analogues thereof comprise at least 5 consecutive nucleotides from the nucleic acid sequences as set in forth in any one of SEQ ID NOs: 1, 2, 3, 5, 6, 11 and 13-19.

12. The pharmaceutical composition of claim 1, wherein the combination of antisense oligonucleotides or analogues thereof enhances the anti-tumour effect of standard doses of the one or more chemotherapeutic agents.

13. A combination of two or more antisense agents complementary to non-contiguous regions of a thymidylate synthase mRNA for use in the treatment of cancer together with a chemotherapeutic agent, wherein the use of the combination reduces the amount of chemotherapeutic required to effectively treat a mammal with cancer.

14. The combination according to claim 13, wherein the combination and chemotherapeutic agent reduce the number of neoplastic cells in said mammal.

15. The combination according to claim 13, wherein the sequences of two of the antisense agents of the combination are selected from the group of SEQ ID NO: 1 and 2, or SEQ ID NO: 1 and 3.

16. The combination according to claim 13, wherein at least one of said two or more antisense agents consists of less than 20 nucleotides.

17. The combination according to claim 16, wherein at least one of said two or more antisense agents is complementary to least 5 consecutive nucleotides from the nucleic acid sequences as set forth in any one of SEQ ID NOs: 1, 2, 3, 5, 6, 11 and 13-19.

18. The combination according to claim 17, wherein the at least one of said two or more antisense agents consists of less than 20 nucleotides.

19. A combination of two or more antisense agents complementary to different regions of a target mRNA for use in the treatment of cancer together with a chemotherapeutic agent, wherein the use of the combination reduces the amount of chemotherapeutic required to effectively treat a mammal with cancer and wherein at least two of said two or more antisense agents have different backbone chemistries.

20. The combination according to claim 19, wherein one or more said two or more antisense agents is an antisense RNA.

21. The combination according to claim 20, wherein the sequence of the antisense RNA is as set forth in any one of SEQ ID NOs: 20-21.

22. A method of treating cancer using a combination of two or more antisense agents complementary to non-contiguous regions of a target mRNA, comprising the step of administering the combination to a mammal in need thereof, wherein the use of the combination reduces the amount of chemotherapeutic required to effectively treat a mammal with cancer and wherein at least one of said two or more antisense agents consists of less than 20 nucleotides.

23. The method according to claim 22, wherein the target mRNA is a thymidylate synthase mRNA.

24. A method of treating cancer using the combination according to claim 19, comprising the step of administering the combination to a mammal in need thereof, wherein enhanced cancer tissue penetration or neoplastic cellular uptake of antisense agent molecules is achieved compared to the administration of antisense agents having the same backbone chemistries and being complementary to the same regions as said antisense agents having different backbone chemistries.

25. The method according to claim 24, wherein the target mRNA is a thymidylate synthase mRNA.
